(19) United States
(12) Patent Application Publication
(10) Pub. No.: US 2023/0167414 A1
(43) Pub. Date: Jun. 1, 2023

(54) METHODS FOR ISOLATING AND CULTURING TUMOR CELLS

(71) Applicant: Georgetown University, Washington, DC (US)

(72) Inventors: Paula R. Pohlmann, Bethesda, MD (US); Seema Agarwal, Alexandria, VA (US); Richard Schlegel, Rockville, MD (US)

(73) Assignee: Georgetown University, Washington, DC (US)

(21) Appl. No.: 17/921,606

(22) PCT Filed: Apr. 26, 2021
(86) PCT No.: PCT/US2021/029244

§ 371 Date: Oct. 26, 2022

Related U.S. Application Data
(60) Provisional application No. 63/015,665, filed on Apr. 26, 2020.

(51) Int. Cl. C12N 5/09; G01N 33/574

(52) U.S. Cl. CPC C12N 5/0693; G01N 33/574; C12N 2501/999; C12N 2513/00; C12N 2500/99; C12N 2500/40; C12N 2500/02

(57) ABSTRACT

Methods of generating a population of tumor cells, such as circulating tumor cells (CTCs) isolated from fluid from a subject. The methods involve collecting a fluid sample containing the tumor cells from the subject, and culturing the tumor cells in the fluid sample in a three-dimensional cell culture, wherein the three-dimensional cell culture comprises at least one inhibitor of Rho-kinase to generate the population of CTCs. If the fluid is whole blood or contains blood, the method may also involve subjecting the fluid sample to density gradient separation to separate the tumor cells from the fluid prior to culturing. In addition, methods of identifying a candidate treatment for a subject having a condition marked by the presence of tumor cells, methods of monitoring in a subject the persistence, regression, or progression of a disease or condition marked by the presence of tumor cells, and methods of generating a cell line of tumor cells.

### CROSS-REFERENCE TO RELATED APPLICATION

This application claims the benefit of priority to U.S. Provisional Application No. 63/015,665, filed on Apr. 26, 2020, which is herein incorporated by reference in its entirety for all purposes.

### STATEMENT REGARDING FEDERALLY SPONSORED RESEARCH OR DEVELOPMENT

This invention was made with government support under grant number P30 CA051008 awarded by the National Cancer Institute of the National Institutes of Health. The government has certain rights in the invention.

### FIELD OF THE INVENTION

The invention is directed to methods of generating populations of tumor cells, such as circulating tumor cells (CTCs), found in a fluid from a subject. The invention is also directed to method of using the populations of tumor cells.

### BACKGROUND

Cancer metastasis is estimated to cause approximately 90% of cancer-associated deaths; however, the mechanistic underpinnings of metastasis are unclear. To this end, circulating tumor cells (CTCs) may be useful in identifying key drivers of metastasis, as well as in predicting responses to cancer therapies.

CTCs are a population of cells that enter the blood stream from the primary tumor and seed distant sites. As revealed by single-cell mutational analyses, CTCs can have novel mutations as compared to primary tumor tissue, which indicate that these cells can be used to elucidate the genetic markers of disease progression and of metastasis, therefore providing potential targets for prognosis and therapy. However, the potential for CTCs to serve as a source of key insights into the metastatic process and the therapeutic response is only realized if these cells can be properly isolated and characterized.

Current methods of isolating CTCs have had limited success. For example, the CellSearch technology, which is the only FDA-approved method for isolating CTCs, uses antibodies to epithelial markers to separate cancer cells from the surrounding hematopoietic cells, but studies have shown that this technology failed to capture the heterogeneity of CTCs. Other methods are based on physical cellular properties such as cell density and size to isolate CTCs, but the mechanical stress to which the CTCs are exposed can lead to poor success rates for culturing cells (6-20% of cultures initiated). Therefore, there is a need for an efficient and effective method of separating CTCs from blood so that they can then be successfully cultured.

Moreover, tumor cells may be present in other fluids besides blood. Thus, there is a similar need for an effective method of separating tumor cells from fluids so that they can be cultured and characterized.

### SUMMARY OF THE INVENTION

An aspect of the present invention relates to methods of generating a population of tumor cells isolated from a fluid from a subject. The methods may comprise collecting a fluid sample containing the tumor cells from the subject; and culturing the tumor cells in a three-dimensional cell culture (3DCC), wherein the 3DCC comprises at least one inhibitor of Rho-kinase (ROCK inhibitor), to generate the population of tumor cells.

The subject may have, or may be suspected to have, abnormal epithelial tissue, such as an epithelial malignancy, in situ tumor, premalignant lesion, or benign lesion. In some embodiments, the cells may be derived from the abnormal epithelial tissue.

In some embodiments, the fluid sample may derive from or be in association with the abnormal epithelial tissue. The fluid sample may be selected from pleural effusion, pericardial effusion, ascites, cerebrospinal fluid (CSF), urine, fluid from ductal lavage, fluid from bronchoalveolar lavage, fluid from endoscopic retrograde cholangiopancreatography (ERCP) duct cytologic brushing, fluid from esophageal brushing, fluid from cervical brushing, fluid from uterine brushing, and fluid from cystic lesions that can be aspirated with a needle.

In embodiments of the invention, the methods may further comprise isolating the tumor cells prior to culturing the tumor cells in the 3DCC. In some embodiments, the tumor cells may be isolated by density gradient separation. In some embodiments, the tumor cells may be isolated by centrifugation.

An aspect of the present invention relates particularly to methods of generating a population of CTCs isolated from blood from a subject. The methods may comprise collecting a whole blood sample from the subject, subjecting the collected whole blood sample to density gradient separation to separate the CTCs from at least erythrocytes in the whole blood sample; and culturing the separated CTCs in a 3DCC, wherein the 3DCC comprises at least one ROCK inhibitor, to generate the population of CTCs. The CTCs may be derived from one or more malignant tumors in the subject. In certain embodiments, the CTCs do not originate from a blood-born cancer. In some embodiments, the whole blood sample is collected in a heparinized collection tube. In some embodiments, the whole blood sample is diluted with a saline solution prior to being subjected to density gradient separation.

The density gradient separation for use in the methods of the invention may comprise a medium. Examples of such mediums may include, but are not limited to, a polysaccharide, colloidal silica particles, a polyhydric alcohol, inorganic salt, and iodinated gradient media. In some embodiments, the medium is FICOLL-PAQUE™. The density gradient separation may separate the tumor cells from free granulocytes in addition to erythrocytes in the whole blood sample.

In some embodiments, the 3DCC comprises a cell scaffold selected from the group consisting of a hydrogel, collagen sponge, gelatin, nanofiber scaffold, polycaprolactone scaffolds, and polystyrene. In other embodiments, the 3DCC comprises a scaffold-free vessel. In certain embodiments, the scaffold-free vessel may inhibit attachment of the cells to the vessel surface.

In some embodiments, the at least one ROCK inhibitor is a small molecule inhibitor of Rho kinase 1 (ROCK 1), a small molecule inhibitor Rho kinase 2 (ROCK 2), a RNAi molecule directed to ROCK 1, a RNAi molecule directed to ROCK 2, or a combination thereof. For example, the at least one ROCK inhibitor may be Y-27632, HA1100 hydrochloride, HA1077, GSK429286, or a combination thereof.

The 3DCC may further comprise adenine. In addition, the 3DCC may further comprise feeder cells. In some embodiments, the 3DCC may be serum-free.

In some embodiments, the 3DCC may comprise oxygen in an amount of less than about 10%, or less than about 5%. In some embodiments, the 3DCC may comprise carbon dioxide in an amount of less than about 10%.

The method of generating a population of cells such as CTCs may not involve a cell-surface marker separation technique, the use of microfluidics, and/or a size-based separation technique.

An aspect of the present invention relates to methods of identifying a candidate treatment for a subject having a condition marked by the presence of tumor cells such as CTCs. The methods may comprise obtaining a population of tumor cells; determining a response profile of at least a portion of the population of the tumor cells; and identifying a candidate treatment for the subject based on the determined response profile. The population of the tumor cells may be obtained by collecting a fluid sample containing the tumor cells from the subject; and culturing the tumor cells in a 3DCC, wherein the 3DCC comprises at least one ROCK inhibitor, to generate the population of tumor cells. The tumor cells may be isolated from the fluid sample prior to culturing the tumor cells in the 3DCC. In embodiments in which the tumor cells are CTCs, the population of the CTCs may be obtained by collecting a whole blood sample from the subject, subjecting the collected whole blood sample to density gradient separation to separate the CTCs from erythrocytes in the whole blood sample, and culturing the separated CTCs in a 3DCC, wherein the 3DCC comprises at least one ROCK inhibitor.

The condition marked by the presence of tumor cells may be one or more neoplasias, hyperplasias, malignant tumors, in situ tumors, and/or benign tumors.

The response profile may be at least partially determined by identifying the sequence of at least one portion of DNA extracted from the tumor cells, by identifying at least one mRNA that is produced in the tumor cells, by identifying at least one mRNA that is not produced in the tumor cells, by identifying one or more proteins that the tumor cells express, by identifying one or more proteins that the tumor cells do not express, or by subjecting the tumor cells to a pharmacologic therapy and determining the therapeutic index of the pharmacologic therapy on the tumor cells. Examples of a pharmacologic therapy include, but are not limited to, one or more chemotherapeutic agents, targeted agents, endocrine agents, immunotherapies, or other drugs non-chemotherapies.

Another aspect of the present invention relates to methods of monitoring in a subject the persistence, regression, or progression of a disease or condition marked by the presence of tumor cells, such as CTCs. The methods may comprise obtaining a population of the tumor cells, assaying at least a portion of the population of tumor cells; and evaluating the results of the assay to determine whether the disease has disappeared, regressed, persisted, or progressed. The at least a portion of the population of tumor cells may be assayed to identify the presence, or the extent to which there is a presence, of a marker of persistence, regression, or progression. The population of the tumor cells may be obtained by collecting a fluid sample containing the tumor cells from the subject; and culturing the tumor cells in a 3DCC, wherein the 3DCC comprises at least one ROCK inhibitor, to generate the population of tumor cells. The tumor cells may be isolated from the fluid sample prior to culturing the tumor cells in the 3DCC. In embodiments in which the tumor cells are CTCs, the population of the CTCs may be generated by collecting a whole blood sample from the subject, subjecting the collected whole blood sample to density gradient separation to separate the CTCs from erythrocytes in the whole blood sample, and culturing the separated CTCs in a 3DCC, wherein the 3DCC comprises at least one ROCK inhibitor;

Further, an aspect of the present invention relates to methods of generating a cell line of tumor cells, such as CTCs, isolated from a subject, the methods comprising collecting a fluid sample containing the tumor cells from the subject, and culturing the tumor cells in the fluid in a 3DCC, wherein the 3DCC comprises at least one ROCK inhibitor. The methods may additionally comprise isolating the tumor cells prior to culturing the tumor cells in the 3DCC. In embodiments in which the tumor cells are CTCs, the method comprise collecting a whole blood sample from the subject, subjecting the collected whole blood sample to density gradient separation to separate the CTCs from erythrocytes in the whole blood sample, and culturing the separated CTCs in a 3DCC, wherein the 3DCC comprises at least one ROCK inhibitor.

An additional aspect of the present invention relates to methods of generating animal models of a disease or condition marked by the presence of tumor cells, such as CTCs, in a subject. The methods may comprise obtaining a population of the tumor cells; injecting a portion of the tumor cells into an animal, such as mice; and obtaining from the animal cells or tumors that are associated with the disease or condition for use in other applications. The population of tumor cells may be obtained by collecting a fluid sample containing the tumor cells from the subject, and culturing tumor cells in a 3DCC, wherein the 3DCC comprises at least one ROCK inhibitor. The tumor cells may be isolated from the fluid sample prior to culturing the tumor cells in the 3DCC. In embodiments in which the tumor cells are CTCs, the population of CTCs may be obtained by collecting a whole blood sample from the subject, subjecting the collected whole blood sample to density gradient separation to separate the CTCs from erythrocytes in the whole blood sample, and culturing the separated CTCs in a 3DCC, wherein the 3DCC comprises at least one ROCK inhibitor. Examples of applications in which the animal cells or tumors associated with the disease or condition may be used include, but are not limited to, identifying a candidate treatment, and generating cell lines for testing.

Yet another aspect of the present invention relates to a cell culture composition comprising (a) a population of tumor cells, such as CTCs, and (b) a 3DCC, wherein the 3DCC comprises at least one ROCK inhibitor. The population of tumor cells may be collected from a fluid sample from a subject.

### BRIEF DESCRIPTION OF THE DRAWINGS

The present disclosure will be further explained with reference to the attached drawing figures.

FIG. 1 depicts the structures of various ROCK inhibitor compounds according to embodiments of the invention.

FIG. 2 depicts results from using the methods of the present invention after 4 days of culture in the 3DCC conditions according to embodiments of the invention.

FIG. 3 depicts Real-time polymerase chain reaction (RT-PCR) followed by sequencing to show the expression of EpCAM, CK5 and CK8 (epithelial origin), mammaglobin (breast origin), and CD45 (neutrophil/leukocyte origin) from RNA of CTC cultures. RT-PCR was used to confirm that RNA isolated from CTCs was of breast and epithelial origin prior to RNA-seq analysis.

FIGS. 4A-4D show CTC successfully propagated breast epithelial cells, as described in Example 1. FIG. 4A shows visual workflow of CTC processing. FIG. 4B shows hormone receptor status of cultured CTCs, based on clinical data. FIG. 4C shows representative phase images (20×) taken of CTCs and healthy donors after 30 days in culture. Red arrows indicate for CTCs and white arrows indicate the adherent leukocyte cells on the top of which CTCs attach and expand. FIG. 4D shows RT-PCR results of RNA from the CTCs, demonstrating that the CTCs were of breast and epithelial origin. FIG. 4e shows a box and whisker plot depicting CD45 relative expression following qPCR amplification. CD45⁻ samples are those that were observed to have decreased propagation with not many leukocytes (if any) attached to the surface of culture dish and did not generate enough high-quality RNA for RNA-sequencing analysis (CTCs 1, 2, 5, 6, 7, and 11).

FIGS. 5A-5D depict different genetic signature for CTCs cultures versus cells from healthy donors (HD) by RNA-sequencing (RNA-seq) analysis, as described in Example 1. FIG. 5A shows a principle-component analyses of gene expression data, which reveals that the five healthy donors (HD) are clustered together, and distinct from all six tested CTC samples. FIGS. 5B and 5C shows that, overall, RNA-seq identified 7,234 significantly differentially expressed genes (DEGs) between CTCs and HDs with an adjusted p-value <0.01, with 3,657 genes upregulated and 3,577 genes downregulated. FIG. 5D shows unbiased CTC isolation as documented by the presence of different genetic signatures, including epithelial and epithelial mesenchymal transition.

FIGS. 6A-6D depict RNA-sequencing (RNA-seq) analysis confirming generated CTCs are malignant, as described in Example 1. FIG. 6A shows use of the Kyoto Encyclopedia of Genes and Genomes (KEGG) computational tool and found the enrichment of 52 pathways (adjusted p-value <0.05). FIG. 6B shows the results of an analysis using the Gene Ontology (GO) project. FIGS. 6C and 6D show the results of an analysis using Gene Set Enrichment Analysis (GSEA).

FIGS. 7A-7F show that cultures propagated CTCs and favored the survival neutrophils compared to healthy donors and depicts the presence of leukocytes from donor supporting the cultures, in particular neutrophils clumped with CTCs at separation, and monocytes, as described in Example 1. FIG. 7A shows Estimating the Proportion of Immune and Cancer cells (EPIC) output of cell types, based on RNA-sequencing data, and considers all cells including CTCs, here labeled as “other” category (pink). The “other” category refers to any non-immune cells (of which CTCs would be a part of). FIG. 7B shows EPIC deconvolution of cell types, without considering the “other” category. This allows for the closer inspection of immune cell types present according to enriched RNA transcripts. FIG. 7C shows CIBERSORT deconvolution results of immune cell types. CIBERSORT utilizes a different method for the identification of immune cells than EPIC. FIG. 7D shows a clustergram of the ImSig macrophage signature. FIG. 7E shows a clustergram of the ImSig neutrophil signature. FIG. 7F shows EPIC deconvolution of cell types, subtracting the “other” category (CTCs). This allows for the closer inspection of immune cell types present, including neutrophils, according to enriched RNA transcripts. HD=healthy donor control sample.

FIGS. 8A-8D depict generation in a mouse of a tumor from injection of CTCs that were obtained from a patient with lung cancer, as described in Example 5. FIG. 8A shows a tumor in situ in the scruff of a mouse. FIG. 8B shows a tumor that has been removed from a mouse. FIG. 8C shows a liver that was harvested from a mouse that had generated a tumor. FIG. 8D shows a spleen that was harvested from a mouse that had generated a tumor.

FIG. 9 shows tumor growth in CTC-derived xenograft (CDX) mouse models, as described in Example 6.

FIG. 10A-10C show analysis of metastases in colon cancer CDX mouse model, as described in Example 6. FIG. 10A shows representative images of primary tumor and metastases from a CDX mouse. FIG. 10B shows Hematoxylin and Eosin (H&E) images of primary tumor and metastases from CDX mouse. FIG. 10C shows mutation in adenomatous polyposis coli (APC) gene present in patient's primary tumor is also present in CDX tumor and metastases as observed by real-time polymerase chain reaction (PCR) followed by Sanger sequencing of the PCR product to confirm the mutation.

### DETAILED DESCRIPTION OF THE INVENTION

The practice of the present invention will employ, unless otherwise indicated, conventional techniques of at least cell biology, cell culture, molecular biology, oncology, and microbiology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

The present invention relates to methods of generating populations of tumor cells separated from fluid from a subject, including CTCs separated from blood, and uses for the tumor cell populations. The invention is based in part on the discovery that the combination of collecting a fluid sample from the subject, and then placing the tumor cells from the fluid sample in a 3DCC comprising at least one ROCK inhibitor, can successfully produce a population of tumor cells that can then be used to study genetic determinants of metastasis and other applications.

For the purposes of the present invention, it is not necessary to know the tissue of origin of the tumor cells to practice the present invention. In fact, in some embodiments, the subject may not have been diagnosed with any sort or tumor or abnormal tissue growth. In these embodiments, it is possible that the methods of the present invention, once applied to the collected fluid sample, would result in no populations of tumor cells. The result of obtaining no populations of tumor cells may still be informative, for example, of an indication as to the stage, activity, or “aggressiveness” of the tumor/cancer or abnormal growth in the subject.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10.

As used herein, the phrase “circulating tumor cells” or “CTCs” is meant to indicate living abnormal cells that have detached into the vasculature or lymphatics from one or more tumors or abnormal growths, such as neoplasias, hyperplasias, malignant or non-malignant tumors, as well as their supporting cells. In one embodiment, the CTCs are derived and detached from malignant tumors. As used herein, CTCs do not include cells originating from blood born cancers, such as leukemia, lymphoma, or myeloma.

### Methods of Generating Tumor Cell Population

An aspect of the present invention relates to a method of generating a population of tumor cells isolated from fluid from a subject. The methods comprise collecting a fluid sample containing the tumor cells from the subject, and culturing the tumor cells in a 3DCC that comprises at least one ROCK inhibitor.

The fluid sample may be any fluid derived from or in association with epithelial tissue. Examples include, but are not limited to, whole blood, pleural effusion, pericardial effusion, ascites, CSF, urine, fluid from ductal lavage, fluid from bronchoalveolar lavage, fluid from ERCP duct cytologic brushing, fluid from esophageal brushing, fluid from cervical brushing, fluid from uterine brushing, and fluid from cystic lesions that can be aspirated with a needle.

The amount of fluid collected may be virtually any amount. In some embodiments, the collected whole blood may be between about 0.1 ml and about 100 ml, such as, for example, about 0.1 ml, 0.2 ml, 0.3 ml, 0.4 ml, 0.5 ml, 1 ml, 2 ml, 3 ml, 4 ml, 5 ml, 6 ml, 7 ml, 8 ml, 9 ml, 10 ml, 11 ml, 12 ml, 13 ml, 14 ml, 15 ml, 16 ml, 17 ml, 18 ml, 19 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90, ml, 95 ml, or 100 ml.

In some embodiments of the invention, the methods may further comprise isolating the tumor cells, such as isolating the tumor cells from the rest of the fluid sample, prior to culturing the tumor cells in the 3DCC. In some embodiments, the tumor cells may be isolated by density gradient separation, for example, if the fluid sample is whole blood or comprises blood (such as if the fluid comprises ascites, or comprises a pleural effusion that is hemorrhagic). In some embodiments, the tumor cells may be isolated by centrifugation, for example, if the fluid sample comprises an effusion, urine, or otherwise large volume.

In some embodiments, if the fluid sample is a low-volume fluid (e.g., less than 0.1 ml), such as small cystic lesion aspirates, bronchoalveolar lavage, biliary or esophageal brushing, or CSF sample, tumor cells in the fluid sample may be isolated by, for instance, centrifugation, or the entire fluid sample may be included for culturing in the 3DCC.

In embodiments in which the fluid sample is a blood sample, the blood sample may be obtained from any region of the subject including, but not limited to, the neck, chest, abdomen, and periphery. In some embodiments, the blood sample may be from the peripheral circulation of the subject. The initial sample collected may be whole blood as is routine and standard in the art. For example, the blood may be collected using vascular access devices known in the art, including, not limited to, a peripherally inserted central catheter, a mediport, and an intracath. In addition, the blood may be collected in standard collection tubes, in which the collection tubes contain heparin, such as but not limited to sodium heparin or lithium heparin. In some embodiments, the collection tubes may not contain ethylenediaminetetraacetic acid (EDTA).

In embodiments in which the fluid sample is a blood sample, typically, about 10 ml to about 100 ml of blood may be collected from the subject in relation to other blood tests. The present invention, however, does not necessarily require up to 100 ml of whole blood. In some embodiments, the collected whole blood may be between about 1 ml and about 10 ml, such as, for example, 1 ml, 2 ml, 3 ml, 4 ml, 5 ml, 6 ml, 7 ml, 8 ml, 9 ml, or 10 ml. In other embodiments, the methods of the present invention may be practiced on more than 100 ml of collected whole blood.

In embodiments in which the fluid sample is a blood sample, the collected whole blood may or may not be diluted with a saline solution. Examples of saline solutions that may be used to dilute the whole blood include, but are not limited to, phosphate buffered saline (PBS), Hank's balanced salt solution (HBSS), Dulbecco's phosphate buffered saline (DPBS), Tri-buffered saline (TBS), Ringer's lactate solution (RLS), Tyrode's balanced salt solution (TBSS), and the like. If the collected blood is diluted, the blood may be diluted in virtually any ratio. In some embodiments, the collected blood may be diluted with saline solution in a volume ratio of about 10:1 to about 1:10, blood:saline solution, or any ratio therebetween. For example, the collected blood may be diluted with saline solution in a blood:saline solution volume ratio of about 10:1, or about 9:1, or about 8:1, or about 7:1, or about 6:1, or about 5:1, or about 4:1, or about 3:1, or about 2:1, or about 1:1, or about 1:2, or about 1:3, or about 1:4, or about 1:5, or about 1:6, or about 1:7, or about 1:8, or about 1:9, or about 1:10.

In embodiments in which the fluid sample contains blood or the fluid sample is whole blood, the fluid sample may be subjected to a density gradient (also known as density gradient centrifugation) to separate at least some components of the blood from one another. In some embodiments, the fluid may be subjected to the density gradient within about four hours of collecting the fluid, such as within about three hours, or within about two hours, or within about one hour, or immediately. In some embodiments, the collected fluid may not be subjected to the density gradient until after a longer period of time after the fluid is collected, for instance, up to about 24 hours, or up to about 48 hours, after the fluid is collected. In embodiments in which the collected fluid is not subject to a density gradient immediately, the collected fluid may be placed on a medium. As an example, the collected fluid may be placed on a medium if the collected fluid must be transported.

The use of density gradient for blood component separation is well-known in the art. In some embodiments, the media of the density gradient may comprise FICOLL™ (neutral, highly-branched polysaccharide). For example, FICOLL-PAQUE™ is a density gradient medium commonly used in blood separation techniques in which the density gradient medium is placed at the bottom of conical tubes and blood is subsequently layered on top and then spun down in a centrifuge. After spinning at the appropriate force for the appropriate length of time, the plasma and peripheral blood mononuclear cells are then separated from free granulocytes and erythrocytes. Separating components of blood using density gradients are well-known in the art.

Other density gradient media that may be used in connection with the claimed methods include, but are not limited to, PERCOLL™ (colloidal silica particles), polyhydric alcohols (sucrose, glycerol, sorbitol), polysaccharides (polysucrose and dextrans), inorganic salts (CsCl, Cs₂SO₄, KBr), and iodinated gradient media (diatrizoate, NYCODENZ®, HISTODENZ®, iodixanol). Similar to using FICOLL™, the density gradient medium is placed in a tube and the blood samples are layered in the tube as well, with the blood being layered above or below the density gradient medium based in routine protocol.

Embodiments of the invention relate particularly to methods of generating a population of CTCs isolated from blood from a subject. These methods may comprise collecting a whole blood sample from the subject, subjecting the blood sample to at least one density gradient separation technique to separate the CTCs from at least erythrocytes in the blood sample, and culturing the separated CTCs in a 3DCC that comprises at least one ROCK inhibitor.

In embodiments in which the tumor cells are CTCs, the CTCs need not be “purified” such that the CTCs are the only components remaining after the separation technique. Instead, “separation,” as used herein may mean that the CTCs are no longer in whole blood. Likely, other components from whole blood, besides CTCs, will be included in the separation layer once the whole blood is processed via a density gradient. Accordingly, the term “separated” is not synonymous with “purified.” The term “isolated,” as used herein, can be a relative term such that one component of whole blood, e.g., CTCs, is “isolated” from another component of whole blood, e.g., erythrocytes. If CTCs are “isolated” from all other cellular components of whole blood, they may be considered to be “purified.”

The methods of the present invention can separate from whole blood individual CTCs, or CTC clusters, i.e., groups of tumor cells that travel together in the bloodstream. CTC clusters may be composed of multiple CTCs clumped together, with or without additional cells such as pericytes, immune cells, neutrophils, macrophages, platelets, and cancer-associated fibroblasts.

In some embodiments, the CTCs may be separated from free granulocytes of the whole blood. The term granulocyte is well-understood in the art and includes such types of cells as neutrophils, eosinophils, basophils, and mast cells. In some embodiments, the CTCs may be separated from the erythrocyte and free granulocyte components of whole blood. The CTCs may not be separated from clumped cells including granulocytes, when present and forming a CTC cluster.

The free granulocyte's band of FICOLL-PAQUE™ gradient centrifugation may also be collected and used in cultures of separated CTCs, to further support their growth and culture's propagation. The free granulocytes may be characterized and studied in parallel with the separated and cultured CTCs from a subject. For example, an attending physician would find information generated by granulocytes and CTC study useful as an indication to continue, to start or to change a therapy for a subject based on presence or absence, or type of granulocytes.

The tumor cells, or in the case of CTCs obtained from the portion of the separation layer from the gradient separation that contains the CTCs, are placed in a 3DCC.

In some embodiments, the separated CTCs may be placed in the 3DCC within about four hours of separating the CTCs from the blood, such as within about three hours, or within about two hours, or within about one hour, or immediately. In some embodiments, the separated CTCs may not be placed in the 3DCC until after a longer period of time after the CTCs are separated, for instance, up to about 24 hours, or up to about 48 hours, after the CTCs are separated.

In embodiments in which the separated CTCs are not placed in the 3DCC immediately, the separated CTCs may be preserved, for example by undergoing cryopreservation utilizing a DMSO-based freezing solution. The cryopreserved cells may then be thawed prior to placing them in the 3DCC. DMSO-based freezing methods are well-known in the art.

As used herein, a 3DCC means that the cells will generally not adhere to or attach to a culture vessel surface, and the cells will generally grow as “floating cells” or will begin forming three-dimensional structures in the cell culture medium, such as but not limited to spheroids. For example, cell culture vessels that do not promote attachment of cells to its surface can be used to generate a 3DCC. As is well-known in the art, 3DCC methods may employ scaffold-based culture vessels that utilize synthetic and/or animal-based scaffolds, such as, but not limited to, hydrogels, collagen sponges, gelatin, nanofiber scaffolds, polycaprolactone (PCL) scaffolds, polystyrene, and the like. In some embodiments, 3DCC may employ scaffold-free vessels that promote the formation of freely-floating cells or cell aggregates in the cell culture medium. The term “scaffold” as used herein in conjunction with cell culture vessels is understood in the art, and, likewise, the term “scaffold-free” is also understood in the art to be a culture vessel that lacks a scaffold promoting cell attachment and in vitro three-dimensional cell growth of the cells into spheroids. Some “scaffold-free” culture vessels may actually contain one or more components on the surfaces that prevent, inhibit, or “discourage” cell attachment, and these culture vessels would be considered “scaffold-free” as used herein. For example, some commercially available culture vessels contain hydrophilic, neutrally charged hydrogels that inhibit non-specific attachment of the cells the surface. These culture vessels, although they may contain hydrogel, do not promote cell attachment and three-dimensional growth in or on the scaffold, promote the formation of freely floating cells or cell aggregates and would thus be considered “scaffold-free” for the purposed of the present invention. In some embodiments, the 3DCC environments utilized in the methods of the present invention may comprise scaffold-free culture vessels. In some embodiments, the 3DCC environments utilized in the methods of the present invention may comprise scaffolded culture vessels.

In embodiments of the invention, the 3DCC may comprise a cell culture medium that comprises at least one ROCK inhibitor. The term ROCK is well understood in the art and means rho associated coiled-coil protein kinase. Rho kinase belongs to the Rho GTPase family of proteins, which includes the Rho, Rac1 and Cdc42 kinases. One of the best characterized effector molecules of Rho is ROCK, which is a serine/threonine kinase that binds to the GTP-bound form of Rho. The catalytic kinase domain of ROCK, which comprises conserved motifs characteristic of serine/threonine kinases, is found at the N-terminus. ROCK proteins also have a central coiled-coil domain, which includes a Rho-binding domain (RBD). The C-terminus is made up of a pleckstrin-homology (PH) domain with an internal cysteine-rich domain. The coiled-coil domain is thought to interact with other α-helical proteins. The RBD, located within the coiled-coil domain, interacts only with activated Rho GTPases, including RhoA, RhoB, and RhoC. The pH domain is thought to interact with lipid mediators such as arachidonic acid and sphingosylphosphorylcholine, and it may play a role in protein localization. Interaction of the pH domain and RBD with the kinase domain results in an auto-inhibitory loop. In addition, the kinase domain is involved in binding to RhoE, which is a negative regulator of ROCK activity.

The ROCK family currently consists of two members, ROCK1 (also known as ROKβ or p160ROCK) and ROCK2 (also known as ROKα). ROCK1 is about 1354 amino acids in length and ROCK2 is about 1388 amino acids in length. The amino acid sequences of human ROCK1 and human ROCK2 are well known. For example, the amino acid sequence of ROCK 1 and ROCK2 can be found at UniProt Knowledgebase (UniProtKB) Accession Number Q13464 and 075116, respectively. The nucleotide sequences of human ROCK1 and ROCK2 can be found at GenBank Accession Number NM_005406.2 and NM_004850, respectively. The nucleotide and amino acid sequences of ROCK1 and ROCK2 proteins from a variety of animals are also well-known and can be found in both the UniProt and GenBank databases.

Although both ROCK isoforms are ubiquitously expressed in tissues, they exhibit differing intensities in some tissues. For example, ROCK2 is more prevalent in brain and skeletal muscle, while ROCK1 is more abundant in liver, testes, and kidney. Both isoforms are expressed in vascular smooth muscle and heart. In the resting state, both ROCK1 and ROCK2 are primarily cytosolic, but are translocated to the membrane upon Rho activation. ROCK activity is regulated by several different mechanisms, thus Rho-dependent ROCK activation is highly cell-type dependent, ranging from changes in contractility, cell permeability, migration, and proliferation to apoptosis. At least 20 ROCK substrates have been identified (see Hu et al. (Expert Opin. Ther. Targets, 2005, 9: 715-736), Loirand et al. (Cir. Res., 2006, 98: 322-334), and Riento et al. (Nat. Rev. Mol. Cell Biol., 2003, 4: 446-456), all of which are incorporated by reference).

The role of ROCK in regulating apoptotic signaling is highly cell-type dependent and stimulus dependent. On the other hand, ROCK has also been associated with mediating cell-survival signals in vitro and in vivo. A ROCK-mediated pro-survival effect has been reported in epithelial cells, cancer cells and endothelial cells, as well as in other cell types. In airway epithelial cells, inhibition with Y-27632 or HA 1077 (also known as fasudil) induces membrane ruffling, loss of actin stress fibers and apoptosis.

As used herein, inhibiting ROCK can mean to reduce the activity, function, or expression of at least one of ROCK1 or ROCK2. The activity, function or expression may be completely suppressed, i.e., no activity, function or expression, or the activity, function or expression may simply be lower in treated versus untreated cells. In general, ROCK phosphorylates LEVI kinase and myosin light chain (MLC) phosphatase after being activated through binding of GTP-bound Rho. One embodiment of the present invention thus involves blocking the upstream pathway of ROCK1 and/or ROCK2, for example GTP-bound Rho, such that ROCK1 and/or ROCK2 is not activated or its activity is reduced over untreated cells. Other upstream effectors include, but are not limited to, integrins, growth factor receptors, including but not limited to, TGF-beta and EGFR, cadherins, G protein coupled receptors and the like. Another embodiment of the present invention thus involves blocking the activity, function, or expression of downstream effector molecules of activated ROCK1 and/or ROCK2 such that ROCK1 and/or ROCK2 cannot propagate any signal or can only propagate a reduced signal over untreated cells. Downstream effectors include, but are not limited to, myosin phosphatase-targeting protein (MYPT), vimentin, LIMK, myosin light chain kinase, NHE1, cofilin, Myosin II and the like. For example, both C3 transferase, a ROCK upstream inhibitor that inhibits the activity of Rho, and blebbistatin, a ROCK downstream inhibitor that inhibits the activity of myosin II, when used in the culture conditions described herein in place of a ROCK inhibitor, affected the cells in such a manner as to allow the cells to bypass differentiation and allow proliferation in vitro.

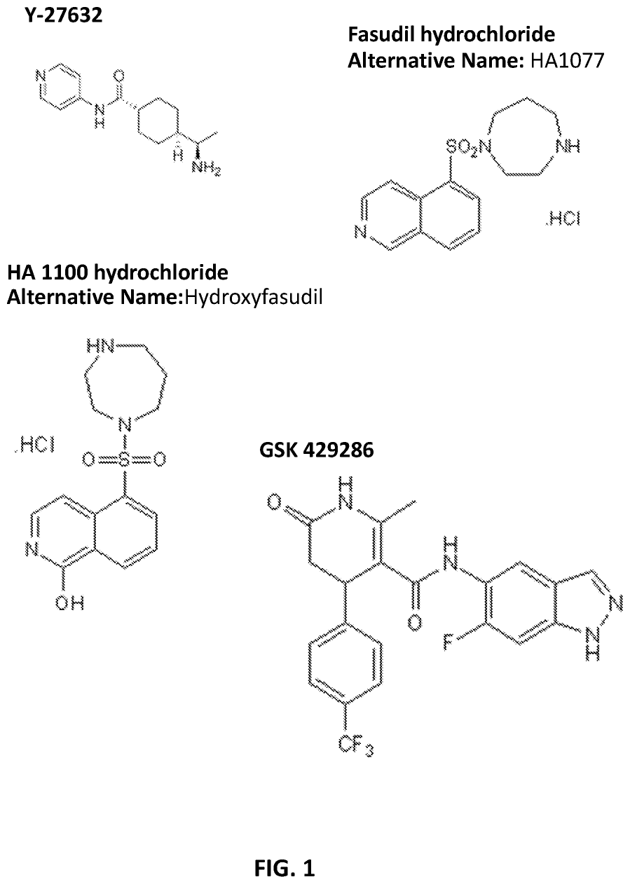

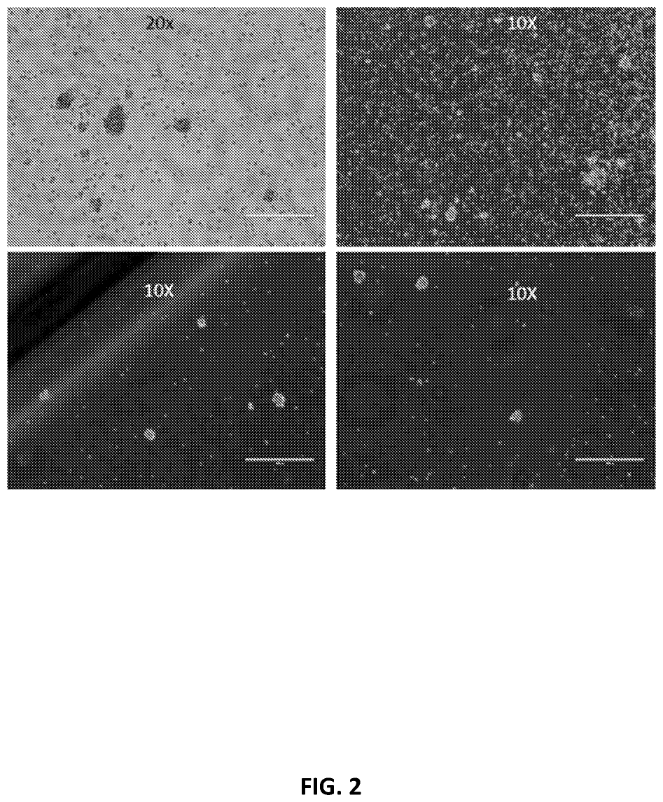

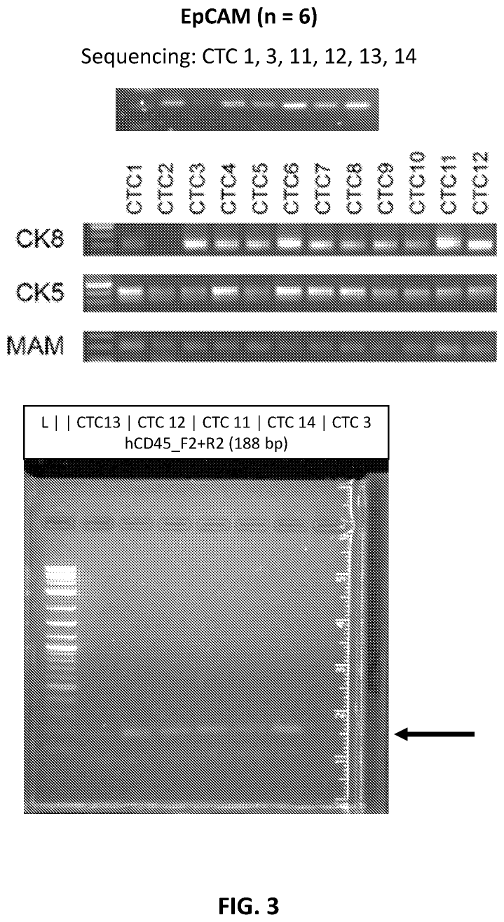

The methods of the present invention comprise inhibiting ROCK while culturing the tumor cells in 3DCCs. In one embodiment, inhibiting ROCK is accomplished by addition of a ROCK inhibitor to the culture medium. Examples of ROCK inhibitors include but are not limited to Y-27632, HA1100, HA1077, Thiazovivin and GSK429286, the structures of which are depicted in FIG. 1. These compounds are well known and commercially available. Additional small molecule Rho kinase inhibitors include but are not limited to those described in PCT Publication Nos. WO 03/059913, WO 03/064397, WO 05/003101, WO 04/112719, WO 03/062225, and WO 03/062227, and described in U.S. Pat. Nos. 7,217,722 and 7,199,147, and U.S. Patent Application Publication Nos. 2003/0220357, 2006/0241127, 2005/0182040 and 2005/0197328, the contents of all of which are incorporated by reference.

Another way of inhibiting ROCK kinase would be through the use of RNA interference (RNAi). RNAi techniques are well known and rely of double-stranded RNA (dsRNA), where one stand of the dsRNA corresponds to the coding strand of the mRNA that codes for ROCK1, and the other strand is complementary to the first strand. The requirements of optimal RNAi species for a given nucleotide sequence are well-known or can be readily ascertained given the state of the art. For example, it is known that optimal dsRNA is about 20-25 nt in length, with a 2 base overhand on the 3′ end of each strand of the dsRNA, often referred to as short interfering RNAs (siRNA). Of course, other well-known configurations such as short hairpin RNA (shRNA) may also work. shRNAs are one continuous RNA strand where a portion is self-complementary such that the molecule is double-stranded in at least one portion. It is believed that the cell processed shRNA into siRNA. The term RNAi molecule, as used herein, is any double stranded double-stranded RNA (dsRNA), where one stand of the dsRNA corresponds to the coding strand of the mRNA that codes for the target gene to be silenced, and the other strand is complementary to the first strand.

Accordingly, some embodiments of the invention involve the use of at least one RNAi molecule and/or at least one antisense molecule in the 3DCC, to inhibit ROCK. In certain embodiments, the RNAi molecule and/or antisense molecule is specific towards ROCK1. In some embodiments, the RNAi molecule or antisense molecule is specific towards ROCK2. In some embodiments, the RNAi molecule and/or antisense molecule is specific towards both ROCK1 and ROCK2. In certain embodiments, at least two RNAi molecules and/or antisense molecules are used, where one is specific towards ROCK1 and the other is specific towards ROCK2.

The RNAi molecules and/or antisense molecules may be part of the cell culture by simply soaking the cells with the naked RNAi molecules and/or antisense molecules as has been reported Clemens et al. (PNAS, 2000, 97: 6499-6503), which is incorporated by reference. The RNAi molecules in the 3DCC and/or antisense molecules may also be part of a complex, such as a liposomal complex that can be used to insert RNAi molecules or antisense/molecules into the separated and cultured tumor cells.

Liposomes fall into two broad classes. Cationic liposomes are positively charged liposomes which interact with the negatively charged dsRNA molecules to form a stable complex. The positively charged dsRNA/liposome complex binds to the negatively charged cell surface and is internalized in an endosome. Due to the acidic pH within the endosome, the liposomes are ruptured, releasing their contents into the cell cytoplasm.

Liposomes that are pH-sensitive or negatively-charged entrap dsRNA rather than complex with it. Since both the dsRNA and the lipid are similarly charged, repulsion rather than complex formation occurs. The dsRNA is thus entrapped in the aqueous interior of these liposomes. pH-sensitive liposomes have been used, for example, to deliver dsRNA encoding the thymidine kinase gene to cell monolayers in culture. One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol. Liposomes that include nucleic acids have been described, for example, in PCT Publication Nos. WO 96/40062 and WO 97/04787, and in U.S. Pat. Nos. 5,264,221 and 5,665,710, all of which are incorporated by reference.

Another type of liposome, a transfersome, is a highly deformable lipid aggregate which is attractive for drug delivery vehicles. Transfersomes may be described as lipid droplets which are so highly deformable that they can penetrate through pores which are smaller than the droplet. Transfersomes are adaptable to the environment in which they are used, for example, they are shape adaptive, self-repairing, frequently reach their targets without fragmenting, and often self-loading. Transfersomes can be made, for example, by adding surface edge-activators, usually surfactants, to a standard liposomal composition.

Another way ROCK1 and/or ROCK2 RNAi can gain access to the tumor cells in the methods of the present invention is through the use of DNA expression vectors that encode the RNAi molecules and/or antisense molecules. Certain embodiments can utilize only one vector, for example when the RNAi molecule is a shRNA, or when opposing promoters are placed on either side there of the coding sequence for the RNAi molecule. Thus “an inhibitor of ROCK” includes the use of DNA that, when transcribed, can block the activity, function, or production of ROCK. The liposomal delivery systems described above are one way in which the DNA encoding an RNAi and/or antisense can enter the cell.

Alternatively, the DNA encoding an RNAi and/or antisense can be prepared in a viral vector system that has the capability of entering into cells. These are well-known in the art and include papovavirus SV40, adenovirus, vaccinia virus, adeno-associated virus, herpes simplex virus (ISV), Epstein-Barr virus (HBV), retrovirus, and baculovirus.

In embodiments of the invention, the 3DCC may comprise a cell culture medium that comprises adenine in addition to a ROCK inhibitor. Adenine is a purine nucleobase whose structure is well-known in the art. The cell culture medium may include any source of adenine, including by not limited to, adenine and compounds that are degraded into adenine such as but not limited to adenosine, adenosine monophosphate, adenosine biphosphate and adenosine triphosphate. The amount of adenine that is added to the 3DCC may vary, but includes ranges of from about 0.1 μg/ml (total culture medium volume) to about 0.2 μg/ml, from about 0.2 μg/ml to about 0.3 μg/ml, from about 0.3 μg/ml to about 0.4 μg/ml, from about 0.4 μg/ml to about 0.5 μg/ml, from about 0.5 μg/ml to about 0.6 μg/ml, from about 0.6 μg/ml to about 0.7 μg/ml, from about 0.7 μg/ml to about 0.8 μg/ml, from about 0.8 μg/ml to about 0.9 μg/ml, from about 0.9 μg/ml to about 1 μg/ml, from about 1 μg/ml to about 1.5 μg/ml, from about 1.5 μg/ml to about 2 μg/ml, from about 2 μg/ml to about 3 μg/ml, from about 3 μg/ml to about 4 μg/ml, from about 4 μg/ml to about 5 μg/ml, from about 5 μg/ml to about 6 μg/ml, from about 6 μg/ml to about 7 μg/ml, from about 7 μg/ml to about 8 μg/ml, from about 8 μg/ml to about 9 μg/ml, from about 9 μg/ml to about 10 μg/ml, from about 10 μg/ml to about 15 μg/ml, from about 15 μg/ml to about 20 μg/ml, from about 20 μg/ml to about 25 μg/ml, from about 25 μg/ml to about 30 μg/ml, from about 30 μg/ml to about 35 μg/ml, from about 35 μg/ml to about 40 μg/ml, from about 40 μg/ml to about 45 μg/ml, from about 45 μg/ml to about 50 μg/ml, from about 50 μg/ml to about 55 μg/ml, from about 55 μg/ml to about 60 μg/ml, from about 60 μg/ml to about 65 μg/ml, from about 65 μg/ml to about 70 μg/ml, from about 70 μg/ml to about 75 μg/ml, from about 75 μg/ml to about 80 μg/ml, from about 80 μg/ml to about 85 μg/ml, from about 85 μg/ml to about 90 μg/ml, from about 90 μg/ml to about 95 μg/ml or from about 95 μg/ml to about 100 μg/ml.

The 3DCC conditions used in the present invention may also comprise low oxygen conditions. As used herein, low oxygen conditions in the 3DCC may mean that the oxygen content in the cell culture incubators is no more than 20% oxygen (v/v). In some embodiments, the 3DCC techniques used in the methods of the present invention may comprise oxygen levels in the incubator at levels of from about 0.5% to about 20%, or from about 0.5% to about 15%, or from about 0.5% to about 10%, or from about 1% to about 10%, or from about 0.5% to less than about 10%, or from about 1% to less than about 10%, or from about 1% to less than about 9%, or from about 1% to less than about 8%, or from about 1% to less than about 7%, or from about 1% to less than about 6%, or from about 1% to less than about 5%, or from about 1% to less than about 4%, or from about 1% to less than about 3%, or from about 1% to less than about 2%. In some embodiments, the oxygen levels used in the 3DCC techniques in the methods of the present invention are about 0.5%, are about 1%, are about 2%, are about 3%, are about 4%, are about 5%, are about 6%, are about 7%, are about 8%, are about 9%, or are about 10%.

The 3DCC conditions used in the present invention may also comprise carbon dioxide. In some embodiments, the 3DCC techniques used in the methods of the present invention may comprise carbon dioxide levels in the incubator at levels (v/v) of from about 0.5% to about 10%, or from about 0.6% to about 10%, or from about 0.8% to about 10%, or from about 1% to about 10%, or from about 0.5% to less than about 10%, or from about 1% to less than about 10%, or from about 1% to less than about 9%, or from about 1% to less than about 8%, or from about 1% to less than about 7%, or from about 1% to less than about 6%, or from about 1% to less than about 5%, or from about 1% to less than about 4%, or from about 1% to less than about 3%, or from about 1% to less than about 2%. In some embodiments, the carbon dioxide levels used in the 3DCC techniques in the methods of the present invention are about 0.5%, are about 1%, are about 2%, are about 3%, are about 4%, are about 5%, are about 6%, are about 7%, are about 8%, are about 9%, or are about 10%.

The 3DCC conditions used in the present invention may also comprise a combination of oxygen and carbon dioxide at the levels described herein. In some embodiments, the 3DCC techniques used in the methods of the present invention may comprise carbon dioxide levels that are greater than oxygen levels. Examples of combinations of carbon dioxide and oxygen in the incubator include embodiments in which the level of carbon dioxide is about 10% and the level of oxygen is about 9%, or about 8%, or about 7%, or about 6%, or about 5%, or about 4%, or about 3%, or about 2%, or about 1%; embodiments in which the level of carbon dioxide is about 9% and the level of oxygen is about 8%, or about 7%, or about 6%, or about 5%, or about 4%, or about 3%, or about 2%, or about 1%; embodiments in which the level of carbon dioxide is about 8% and the level of oxygen is about 7%, or about 6%, or about 5%, or about 4%, or about 3%, or about 2%, or about 1%; embodiments in which the level of carbon dioxide is about 7% and the level of oxygen is about 6%, or about 5%, or about 4%, or about 3%, or about 2%, or about 1%; embodiments in which the level of carbon dioxide is about 6% and the level of oxygen is about 5%, or about 4%, or about 3%, or about 2%, or about 1%; embodiments in which the level of carbon dioxide is about 5% and the level of oxygen is about 4%, or about 3%, or about 2%, or about 1%; embodiments in which the level of carbon dioxide is about 4% and the level of oxygen is about 3%, or about 2%, or about 1%; embodiments in which the level of carbon dioxide is about 3% and the level of oxygen is about 2% or about 1%; and embodiments in which the level of carbon dioxide is about 2% and the level of oxygen is about 1%.

The 3DCC culture conditions used in the present methods may also be serum-free conditions. The term “serum” is well understood in the art of cell culturing and is the component of blood that does not include blood cells or clotting factors, i.e., serum generally is the cell-free component of blood that includes all proteins and other components that do not participate in clotting. Typically, serum from bovine or other animal source is used in cell culture systems. As used herein, “serum-free” is used to mean that the methods cannot include serum from an animal source. Synthetic media that act as “serum replacements” are not from animal sources, thus the term “serum-free” as used herein can include the use of synthetic serum replacement cell culture media components commercially available. In some embodiments, however, the methods of the present invention do not include serum replacement media supplements. In certain embodiments, the methods of the present invention include serum replacement media supplements.

The basal medium used in the methods of the present invention may be any aqueous-based medium and can include any “classic” media such as, but not limited to DMEM (Dulbecco's Modified Essential Medium), Ham's F12 medium, Ham's F-10 medium, RPMI 1640, Eagle's Basal Medium (EBM), Eagle's Minimum Essential Medium (MEM), HEPES, Medium 199 and the like. The culture medium may also be combinations of any of the classical medium, such as but not limited to, a combination of DMEM and F12 Media.

Additional ingredients may be added to the culture medium used in the methods of the present invention. Such additional ingredients may include, but are not limited to, amino acids, vitamins, inorganic salts, ethanolamine, D-glucose, heparin, N-[2-hydroxyethyl]piperazine-N′-[2-ethanesulfonic acid] (HEPES), hydrocortisone, insulin, lipoic acid, phenol red, phosphoethanolamine, putrescine, sodium pyruvate, triiodothyronine (T3), thymidine and transferrin. Alternatively, insulin and transferrin may be replaced by ferric citrate or ferrous sulfate chelates. Each of these additional ingredients is commercially available.

Amino acid ingredients which may be included in the media of the present invention may include, but are not limited to, L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine.

Vitamin that may be added include, but are not limited to, biotin, choline chloride, D-Ca⁺²-pantothenate, folic acid, i-inositol, niacinamide, pyridoxine, riboflavin, thiamine, and vitamin B12.

Inorganic salt ingredients which may be added may include, but are not limited to, calcium salt (e.g., CaCl₂), CuSO₄, FeSO₄, KCl, a magnesium salt, e.g., MgCl₂, a manganese salt, e.g., MnCl₂, sodium acetate, NaCl, NaHCO₃, Na₂HPO₄, Na₂SO₄ and ions of the trace elements selenium, silicon, molybdenum, vanadium, nickel, tin, and zinc. These trace elements may be provided in a variety of forms, such as in the form of salts, e.g., Na₂SeO₃, Na₂SiO₃, (NH₄)₆Mo₇O₂₄, NH₄VO₃, NiSO₄, SnCl, and ZnSO.

Additional ingredients include, but are not limited to, heparin, epidermal growth factor (EGF), at least one agent increasing intracellular cyclic adenosine monophosphate (cAMP) levels, and at least one fibroblast growth factor (FGF). Heparin, EGF, the cAMP-increasing agent(s) and FGF(s) may be added to the basal medium or they may be admixed in a solution of, for example, Dulbecco's Phosphate Buffered Saline (DPBS) and stored frozen until being added to basal medium to formulate the medium to be used in the methods of the present invention.

Heparin may be obtained commercially. Heparin is added to the present media primarily to stabilize the activity of the growth factor components, for example FGF. If heparin is used, it may be added to the basal medium at a concentration of about 1-500 U.S.P. units/liter. EGF is available commercially. If EGF is used, it may be added to the basal medium at a concentration of about 0.00001-10 mg/L.

A variety of agents that increase intracellular cAMP levels may be used in formulating the media of the present invention. Included are agents which induce a direct increase in intracellular cAMP levels, e.g., dibutyryl cAMP, agents which cause an increase in intracellular cAMP levels by an interaction with a cellular G-protein, e.g., cholera toxin and forskolin, agents which cause an increase in intracellular cAMP levels by acting as agonists of β-adrenergic receptors, e.g., isoproterenol and agents which cause an increase in intracellular cAMP levels by inhibiting the activities of cAMP phosphodiesterases, e.g., isobutylmethylxanthine (IBMX) and theophylline. These cAMP-increasing agents are available commercially.

The 3DCC techniques used in the methods of the present invention may or may not include the use of feeder cells. In some embodiments, the methods of the present invention do not include the use of feeder cells. In some embodiments, the methods of the present invention include the use of feeder cells. As used herein, the term “feeder cells” is used herein as it is in the art. Namely, feeder cells are cells that are co-cultured with other cells, i.e., the tumor cells. As used herein, “culturing with tumor cells” means that the feeder cells are cultured sharing the same medium and sharing the same vessel with the tumor cells. Thus, the feeder cells need not be in direct contact with the tumor cells and, for example, can be physically separated from the tumor cells, e.g., by a porous filter, although both sets of cells are in the same vessel sharing the same medium. In some embodiments, the feeder cells are non-proliferating feeder cells. In some embodiments of the present invention, the feeder cells can be treated to inhibit proliferation of the feeders, while still keeping them alive and metabolically active. For example, feeder cells can be irradiated with gamma irradiation and/or treated with mitomycin C, which will arrest cell division but maintain the cells in a metabolically active state. Methods of treating cells to arrest cell division but maintain a metabolically active state are well-known in the art. In certain embodiments, the feeder cells have not been treated to inhibit proliferation. For example, feeder cells, placed on a porous filter that prevents physical contact with the tumor cells, can be cultured with the tumor cells without the need to treat the feeder cells to inhibit their proliferation

Feeder cells can be from any mammal and the animal source of the feeder cells need not be the same animal source as the tumor cells being cultured. For example, feeder cells may be, but are not limited to mouse, rat, canine, feline, bovine, equine, porcine, non-human and human primate feeder cells. The types of feeder cells used are typically spleenocytes, macrophages thymocytes and/or fibroblasts. In one embodiment, the spleenocytes, macrophages thymocytes and/or fibroblasts have been treated such that they are non-proliferating. One example of a feeder cell that may be used in the methods of the present invention is a population of J2 cells. The J2 cells are a subclone of mouse fibroblasts derived from the established Swiss 3T3 cell line. In one embodiment, the J2 cells are gamma irradiated. In another embodiment, the J2 cells are treated with mitomycin C.

In some embodiments, medium conditioned with feeder cells may be used in place of culturing feeder cells with the tumor cells. In some embodiments, the methods of the present invention do not include the use of feeder cell conditioned medium. In some embodiments, the methods of the present invention include the use of feeder cell conditioned medium. Preparing conditioned medium is routine in the art. Generally, preparation of conditioned medium involves culturing cells in a medium, e.g., F-medium as defined herein, for a few days and collecting this medium. The conditioned medium is often, but need not be, combined with fresh medium in a diluted fashion. Discovering the optimal dilution ratios of conditioned medium to “fresh medium” is routine, but the ratios can be from about 1:99 to about 99:1 of “conditioned medium” to “fresh medium.” As used herein, “conditioned medium” is any medium where all or a percentage of the medium has been previously used in culture.

In some embodiments, feeder cell extract can be added to the medium in place of feeder cells themselves. In some embodiments, the methods of the present invention do not include the use of feeder cell extract. In certain embodiments, the methods of the present invention include the use of feeder cell extract. Methods of preparing feeder cell extract are common and are described in Graham et al. (Biochem. 1, 1979, 182: 157-164), Graham (Biochem. 1, 1972, 130: 1113-1124), and Dickson et al. (Proc. Nat'l Acad. Sci., U.S.A., 1983, 80: 5335-5339) all of which are incorporated by reference herein. Discovering the optimal dilution feeder cell extract to medium is routine, but the ratios can be from about 1:99 to about 99:1 of extract to medium.

In embodiments of the present invention in which the tumor cells are CTCs, the methods described herein do not involve the use of cell-surface marker separation, such as the use of antibodies, to separate CTCs from blood. An example is the Cell Search technology that relies on antibodies to EpCAM⁺ and CD45⁻ so that the epithelial cells (EpCAM⁺) can be harvested and leukocytes (CD45⁻) can be removed.

In embodiments of the present invention, the methods described herein do not involve the use of microfluidics to separate CTCs from blood, including methods that may separate cells based on physical cellular properties such as cell density and size.

In some embodiments of the present invention, the methods described herein do not involve the use any size-based separation techniques to separate CTCs from blood.

In embodiments of the invention, the tumor cells are derived from cancers that arise from epithelial tissues. Examples of such cancers include, but are not limited to, bladder cancer, breast cancer, cervical cancer, colorectal cancer, gastric cancer, head and neck cancer, liver cancer, lung cancer, oral cavity cancer, esophageal cancer, biliary cancer, gallbladder cancer, uterine cancer, ovarian cancer, and pancreatic cancer. In some embodiments, the tumor cells are derived from precursor lesions of the cancers that arise from epithelial tissues.

### Methods of Using the Cultured Tumor Cells

Aspects of the invention relate to methods involving a population of tumor cells, such as CTCs, generated by the methods of the present invention described herein.

Embodiments of the present invention relate to methods of identifying candidate treatments for a subject having a condition marked by the presence of tumor cells. The methods may comprise generating a population of tumor cells from fluid collected from the subject in accordance with the methods of the present invention, determining a response profile of at least a portion of the population of tumor cells, and identifying a candidate treatment for the subject based on the determined response profile. In particular embodiments, the methods may comprise generating a population of CTCs from blood collected from the subject in accordance with the methods of the present invention, determining a response profile of at least a portion of the population of CTCs, and identifying a candidate treatment for the subject based on the determined response profile. Conditions marked by the presence of tumor cells may include, but are not limited to, having one or more neoplasias, hyperplasias, malignant tumors, and/or benign tumors.

The response profile, as used herein, may be a collection of one or more data points that would indicate, e.g., to a clinician, the likelihood that a particular treatment will produce a desired response in the subject. A “response” as used in connection with a response profile may or may not be either cell death by any means (necrosis, toxicity, apoptosis, etc.) or a reduction of the growth rate of the population of cultured tumor cells. The response profile need not predict a response with 100% accuracy. A response profile can be a single datapoint or it can be a collection of data.

Any method can be used to identify or determine the response profile of a given population of tumor cells. For example, the response profile may be assessed by sequencing at least part of the DNA or RNA that is isolated from the tumor cells. This may be particularly useful when, for example, it is suspected that a virus, e.g., human papilloma virus (HPV), human immunodeficiency virus (HIV) or other virus that may be causing the abnormal condition. It is not necessary that all of the DNA/RNA be sequenced to provide at least one data point for the response profile. For example, using well-known techniques involving polymerase chain reaction (PCR), it would currently be a matter of simple procedure to use PCR primers with sequences specific for the DNA/RNA suspected of being present, e.g., HPV or HIV, in a PCR reaction to determine if a product is made. If no detectable product is generated after the PCR reaction using specific primers, it may be possible to conclude that the portion of the virus for which the PCR primers are specific may not be present. Likewise, determining the absence of a particular DNA/RNA sequence could also be a data point in a response profile. In this manner, the DNA or RNA is “sequenced” for the purposes of the present invention, although the precise sequence is not determined for the entire DNA/RNA sequence isolated from the cells. Thus, “sequencing” as used herein may or may not result in generating the entire nucleotide sequence of the isolated DNA/RNA. Other methods can also be used to determine the sequence of the isolated DNA/RNA such as, but not limited to Southern blots, Northern blots, RT-PCR, automated sequencing, and the like. Methods of sequencing DNA/RNA are well known in the art and need not be repeated herein.

Similarly, the response profile may be assessed by identifying the presence or absence of at least a portion of one mRNA that may be produced in the tumor cells in vitro. Like determining the sequence of the DNA/RNA above, the precise sequence of the mRNA need not be determined for the entire mRNA isolated from the cells. Methods that can also be used to determine the presence or absence of the sequence of the isolated mRNA include but are not limited to Northern blots, RT-PCR, automated sequencing, and the like. Methods of identifying the presence or absence of the at least one mRNA are well known in the art and need not be repeated herein.

Similarly, the response profile may be assessed by identifying the presence or absence of at least a portion of one protein that may be produced in the tumor cells in vitro. Like determining the sequence of the DNA/RNA above, the precise amino acid sequence of the present or absent protein need not be determined for the entire protein. Methods that can also be used to determine the presence or absence of the sequence of the isolated protein include but are not limited to Western blots, immunohistochemical methods, immunofluorescence methods, ELISA methods, proteomics, and the like. Methods of identifying the presence or absence of the at least one protein are well known in the art and need not be repeated herein. The presence or absence of a protein, e.g., a receptor, may indicate that the cells are susceptible to a particular treatment that may, for example, result in cell death.

The response profile may be assessed by subjecting the tumor cells in vitro to a pharmacologic therapy and determining the response of the cells to the pharmacologic therapy. As used herein, a pharmacologic therapy is not limited to traditional cancer treatments but is used to indicate a therapeutic treatment of any kind using a chemical entity. Examples of a pharmacologic therapy include, but are not limited to, one or more chemotherapeutic agents, targeted agents, endocrine agents, immunotherapies, or other drugs non-chemotherapies. In some embodiments, the response to the pharmacologic therapy can be assessed by determining the therapeutic index of the pharmacologic therapy on the tumor cells. Determining the therapeutic index is common in the art and is simply the ratio of the LD₅₀/EC₅₀, with the LD₅₀ representing the median lethal dose and the EC₅₀ representing the half maximal dose of the pharmacologic therapy on the tumor cells. Other methods of assessing a response to the pharmacologic therapy include, but are not limited to, determining dose response curves, cell survival curves and the like. In some embodiments, the pharmacologic therapy that is used to determine the response of the tumor cells to the pharmacologic therapy can be the same or a different pharmacologic therapy that is later administered to the subject.

Embodiments of the present invention are directed to methods of monitoring the persistence, regression, or progression of a disease or condition, or treatment of a disease or condition, in a subject, in which the disease or condition is marked by the presence of tumor cells. The methods may comprise generating a population of tumors cells from fluid collected from the subject in accordance with the methods of the present invention, assaying at least a portion of the population of the tumor cells, and evaluating the results of the assay to determine whether the disease has disappeared, regressed, persisted, or progressed. In particular embodiments, the methods may comprise generating a population of CTCs from blood collected from the subject in accordance with the methods of the present invention, assaying at least a portion of the population of CTCs, and evaluating the results of the assay to determine whether the disease has disappeared, regressed, persisted, or progressed. As used herein, the phrase “monitor the persistence, regression, or progression” may be used to indicate that the disease or condition in the subject is being periodically checked to determine if the disease or condition is persisting, i.e., remaining static or persisting (no detectable change), regressing (improving), or progressing (worsening).

Conditions marked by the presence of tumor cells may include, but are not limited to, one or more neoplasias, a hyperplasias, malignant tumors, in situ tumors, or benign tumors. In embodiments of the invention, the conditions marked by the presence of tumor cells may include cancers that arise from epithelial tissues, or precursor lesions thereof. Examples of such cancers include, but are not limited to, bladder cancer, breast cancer, cervical cancer, colorectal cancer, gastric cancer, head and neck cancer, liver cancer, lung cancer, oral cavity cancer, esophageal cancer, biliary cancer, gallbladder cancer, uterine cancer, ovarian cancer, and pancreatic cancer.

The tumor cells may be assayed to identify the presence, or the extent to which there is a presence, of various markers of persistence, regression, or progression. These markers may be assessed by, and the tumor cells may be assayed by, sequencing at least part of the DNA or RNA that is isolated from the tumor cells, identifying the presence or absence of at least a portion of one mRNA that may be produced in the tumor cells, and/or identifying the presence or absence of at least a portion of one protein that may be produced in the tumor cells.

The methods of monitoring may be used in conjunction with other monitoring methods or treatment regimens for an abnormal condition and to monitor the efficacy of these treatments. Thus, “monitoring persistence, regression, or progression” may also indicate assessing the efficacy of a treatment regimen by periodically obtaining a population of tumor cells from the subject, assaying a least a portion of the population of tumor cells, and evaluating the results of the assay by correlating any differences in the subject over time with the persistence, regression, or progression of the disease or condition. For example, the methods of the present invention may be used to monitor a subject receiving treatment for a disease, such that a population of tumor cells may generated at multiple time points during the therapy and the populations may be assayed and evaluated to determine if/how the presence of certain biomarkers change over time. The treatment may be maintained if the disease is persisting (for example, if the disease is known to be aggressive and stasis is considered as a successful outcome) or regressing, or the treatment may be changed if the disease is persisting (for example, if stasis is not considered as a successful outcome) or progressing. If the disease is cancer in particular, the methods of the present invention may be used to assess the risk of clinical recurrence, tumor progression or to assess the degree of malignancy, which can be informative as to whether continue a therapy, or to start or to change a therapy. Importantly, the presence or absence of any tumor cells being generated by the methods of invention may be indicative on whether a therapy is successful.

The methods of monitoring can also be used to determine a suitable follow up therapeutic regimen, after an initial treatment. For example, after an initial treatment a population of tumor cells may be obtained from the subject and assayed to determine if the genetic makeup or phenotype of the tumor cell is sufficiently different enough to warrant a new therapy. Thus, in some embodiments, the present invention provides methods of individualizing a therapeutic regimen.

Another aspect of the present invention relate to methods of developing a tumor-cell cell line. The methods may comprise generating a population of tumor cells from fluid collected from a subject having a disease or condition in accordance with the methods of the present invention. These cells lines may then be used for applications in drug discovery, including injecting cells from the cell line into animals to create an animal model for evaluating the disease or condition and for testing candidate therapies.

An additional aspect of the present invention relates to methods of generating animal models of a disease or condition marked by the presence of tumor cells in a subject. The methods may comprise generating a population of the tumor cells from fluid collected from the subject in accordance with methods of the present invention; injecting a portion of the tumor cells into an animal; and obtaining from the mice cells or tumors that are associated with the disease or condition for use in other applications. Examples of such applications include, but are not limited to, identifying a candidate treatment, and generating cell lines for testing.

In some embodiments, animals that may be used to generate the models include, but are not limited to, rodents such as mice and rat, cats, dogs, rabbits, pigs, sheep, goats, cattle, chickens, and non-human primates.

In some embodiments, applications for use with the cells or tumors generated from the animal models include, but are not limited to, identifying a candidate treatment, and generating cell lines for testing. With this in mind, in some embodiments, the methods of identifying a candidate treatment for a subject having a condition marked by the presence of tumor cells as described herein may include a step of generating an animal model of the disease or condition. For example, the methods of identifying a candidate treatment for a subject having a condition marked by the presence of tumor cells may comprise obtaining a population of the tumor cells; generating an animal model of the disease or condition; obtaining from the animal model cells or tumors that are associated with the disease or condition; determining a response profile of at least a portion of the cells or tumor obtained from the animal model; and identifying a candidate treatment based on the determined response profile.

In some embodiments, methods of generating a cell line for research on a disease or condition marked by the presence of tumor cells may comprise obtaining a population of the tumor cells from a subject having the disease or condition marked by the presence of tumor cells; generating an animal model of the disease or condition; obtaining from the animal model cells or tumors that are associated with the disease or condition; and then testing the cells or tumors.

In other aspects of the invention, the populations of tumor cells generated according to the methods of the present invention may also be used in cell-cell interaction assays, including but not limited to cell-cell interactions with fibroblasts, adipocytes, osteoblasts, osteoclasts, hepatocytes, pneumocytes, microglia, and immune cells. The origin of these cells could be the same tumor cell subject, other subjects, established cell lines. The cell lines could be human or animal in origin. The cell-cell interaction assays could be useful in choosing an effective, or perhaps just more effective, therapeutic regimen, including immunotherapy. Cell-cell interaction assays could also be useful in preclinical research as part of drug development efforts or in drug resistance studies.

### Methods of Characterizing Abnormal Tissue Growths

Aspects of the invention relate to methods of characterizing abnormal tissue growths. For example, embodiments of the present invention relate to methods of determining whether an abnormal tissue growth is malignant or benign, or determining the type of malignancy. These methods comprise collecting a fluid sample from the abnormal tissue growth or in association with the abnormal tissue growth from the subject, culturing cells in the fluid sample that are from the abnormal tissue growth in a 3DCC that comprises at least one ROCK inhibitor to generate a population of the cells from the abnormal tissue growth; and analyzing the population of cultured cells from the abnormal tissue growth. The analysis may be to determine, for example, if the abnormal tissue growth is malignant or benign, the type of malignancy, etc.

The analysis of the cultured cells from the abnormal tissue growth may use techniques known in the art for identifying cells as cancerous, for example, histochemistry, immunohistochemistry, electron microscopy, flow cytometry, and image cytometry.

In some embodiments in which there is blood in the fluid sample, or the fluid sample is whole blood, the method may additionally comprise subjecting the sample to at least one density gradient separation technique to separate the cells from the abnormal tissue growth.

In some embodiments, it may be determined that the cells from the abnormal tissue growth in the fluid are not tumor cells if culturing the cells from the abnormal tissue growth does not generate a population of the cells.

### Cell Culture Compositions

Additional aspects of the invention relate to cell culture compositions that comprise (a) a population of tumor cells, and (b) a 3DCC, wherein the 3DCC comprises at least one ROCK inhibitor. In some embodiments, the population of tumor cells is collected from a fluid sample from a subject. In preferred embodiments, the tumor cells are CTCs and are collected from a whole blood sample.

In some embodiments, the population of tumor cells are generated in accordance with the methods of the present invention. In some embodiments, the 3DCC is in accordance with the methods of the present invention.

### EXAMPLES

### Example 1

A study was conducted to isolate, propagate, and characterize short-term CTC cultures from the blood of 12 metastatic breast cancer patients, in accordance with embodiments of the present invention.

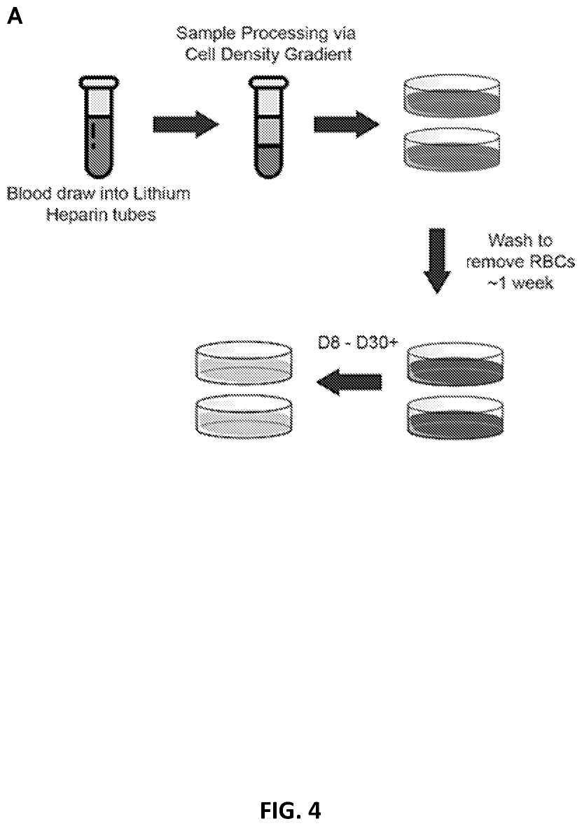

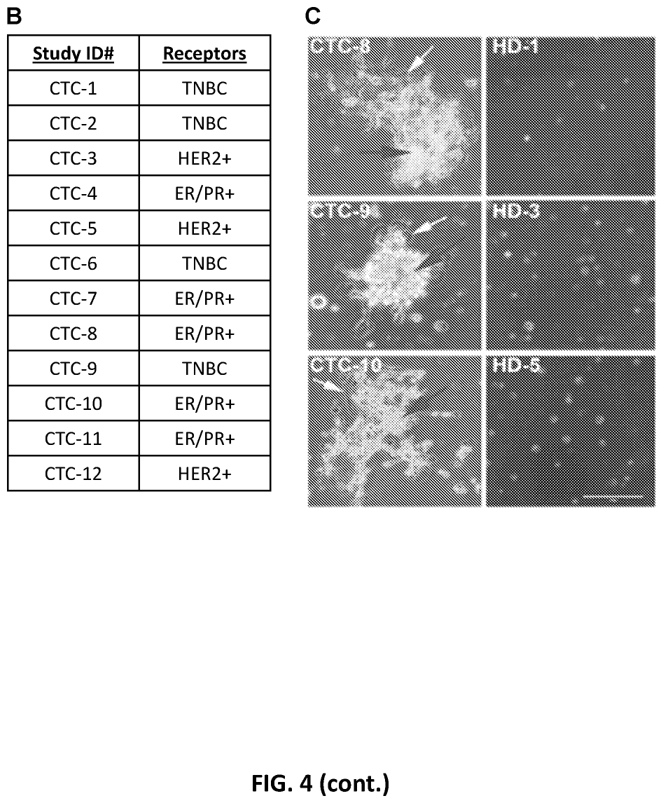

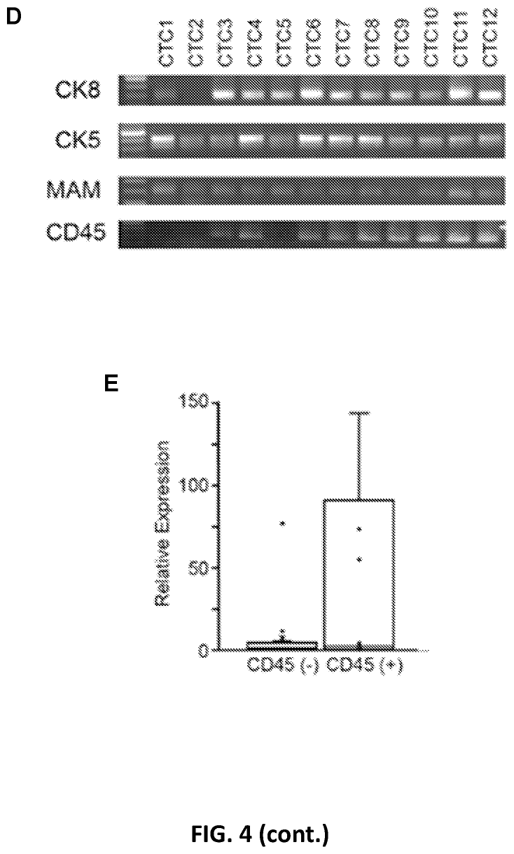

The patients enrolled in the study span the following histological categories: (1) HER2 positive (n=3); (2) triple-negative breast cancer (TNBC) (n=4); and (3) estrogen receptor positive, progesterone receptor positive, and HER2 negative (ER/PR+) (n=5) (see FIG. 4B). An additional five healthy donors (HD) with no known health conditions at the time of consent were also enrolled and used as controls for this study.

The process of isolating the CTCs and preparing the cell cultures is summarized in FIG. 4A. Blood was collected from each patient in a lithium heparin vial, and the blood samples were processed using FICOLL-PAQUE™ PLUS density gradient. The blood was mixed in a 1:1 solution with HBSS, and then split into two vials that each containing FICOLL-PAQUE™. The vials were centrifuged at 400 g for 40 min. After centrifugation, most of the RBCs were pelleted underneath the FICOLL-PAQUE™ Above the FICOLL-PAQUE™ was a buffy coat, formed of mononuclear cells (including CD45⁺ leukocytes), and plasma. Both the buffy coat and plasma were moved to fresh tubes, HBSS was added, and the solution was centrifuged at 400 g for 20 min at 25° C. Following centrifugation, cells were pelleted at the bottom of the tube. The supernatant was aspirated, and three additional washes (once with HBSS, twice with PBS) were conducted using a centrifuge set to 400 g for 10 min each. Upon completion of the final wash, cells were resuspended in culture medium and plated in an ultra low-attachment plate.

Healthy donor samples were processed in a similar manner to the CTC samples. Two tubes of 7.5 mL blood each were drawn from each patient into a lithium heparin vial and the samples were processed using a FICOLL-PAQUE™ PLUS density gradient as discussed above for the processing of the CTCs. Prior to plating of cells on an ultra low-attachment plate, about 20% of cell pellet was spun and processed into RNA using an RNAqueous-Micro Total RNA isolation kit according to manufacturer protocol. The remaining cells were plated in an ultra low-attachment plate.

The HD cells and CTCs were grown in ultra low-attachment plates in hypoxic conditions (5% CO₂ and 2% O₂) at 37° C. in a 3D medium. The 3D medium consisted of Advanced DMEM/F12 supplemented with B27 supplement, 20 ng/mL epidermal growth factor, 20 ng/mL basic fibroblast growth factor, 50 μg/mL heparin, 10 μM of rho-kinase inhibitor Y-27632, adenine, L-glutamine (1×), and antibiotic-antimycotic (1×). Cultures were supplemented with fresh medium every 3 days and washed every 6 days by centrifugation of the supernatant at 100 g for 4 min at 4° C. Cultures were processed for RNA isolation one month after harvesting.

Total RNA isolation was performed using RNAqueous-Micro Total RNA isolation Kit according to manufacturer protocol. RNA were converted to cDNA and PCR was performed on cDNA to test for the presence of epithelial (CK8, CK18), mesenchymal (VIM, SERP), and breast markers (MAM). The PCR products were then sequenced and analyzed.

Of the CTC cultures, six of the 12 cultures exhibited a higher growth potential, i.e., could be cultured for >30 days. These cultures became adherent to the plates and were associated with CD45⁺ cells (see FIG. 4C, left panel; and FIGS. 4D and 4E). None of the HD samples resulted in viable cultures (see FIG. 4C, right panel), even though the HD cells were processed in an identical manner to the samples from patients.

After 30 days, RNA was obtained from all 12 cultures. All samples tested positive for cytokeratin 5 and 8, which are luminal and basal epithelial markers of breast cancer, and tested positive for mammaglobin, which is a biomarker for breast cancer (see FIG. 4D).

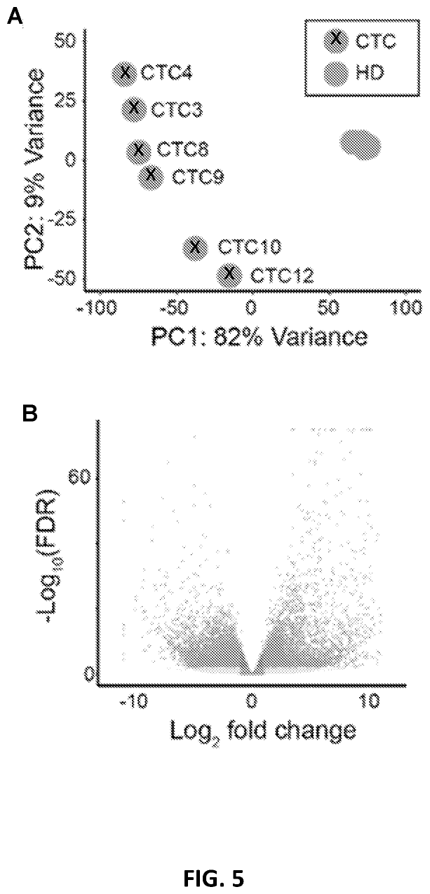

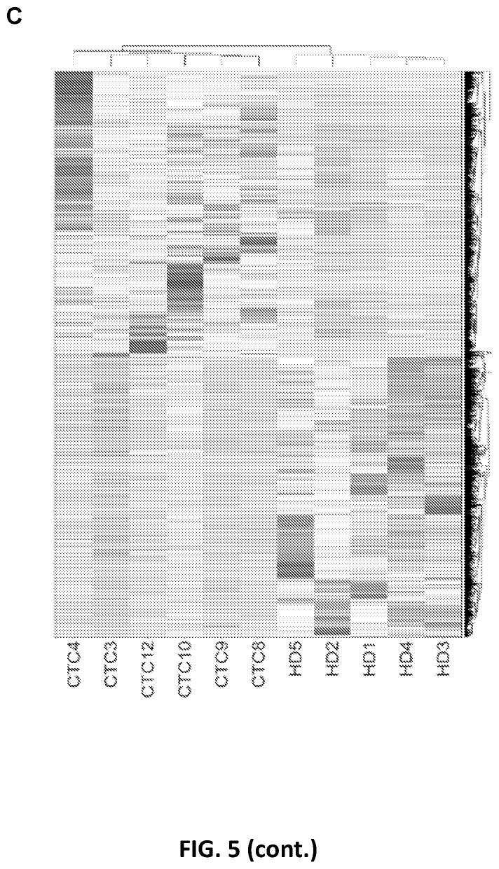

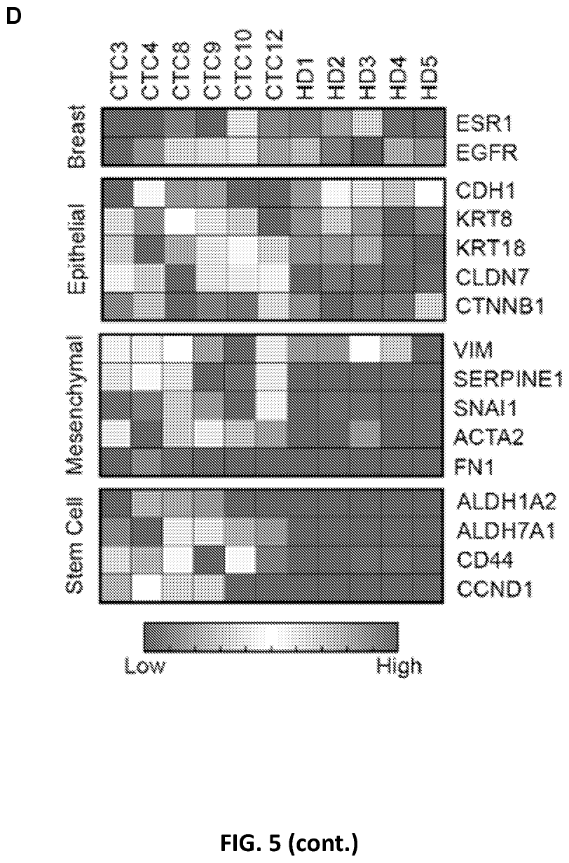

For characterization of the gene expression within the CTCs, only the six samples that could be propagated for over 30 days yielded sufficient quantities of RNA for analysis. These samples were the CTCs that associated with CD45⁺ (see Figures FIGS. 4D and 4E). RNA from the five HDs (harvested from the buffy coat) were also prepped and sequenced, as these samples represent a background of leukocytes in the blood. A principle-component analyses of gene expression data revealed that the 5 HDs are clustered together, and distinct from all 6 CTC samples (see FIG. 5A). CTCs demonstrated no obvious clustering based on hormone receptor status. Overall, the analysis identified 7,234 significantly differentially expressed genes (DEGs) between CTCs and HDs with an adjusted p-value <0.01, with 3,657 genes upregulated and 3,577 genes downregulated (see FIGS. 5B and 5C).

A panel of genes associated with breast, epithelial, mesenchymal, and stem cells were examined. Epithelial markers (KRT8; KRT18; CLDN7; CTNNB1) and mesenchymal markers (VIM; SERPINE1; SNAI1; ACTA2) were upregulated in CTC samples as compared to the HD samples (see FIG. 5d). Cancer stem cell markers ALDH1A2, ALDH7A1, CD44, and CCND1 were also upregulated in CTCs compared to HDs (see FIG. 5D). These results indicate that the methods used in this study propagate CTCs.

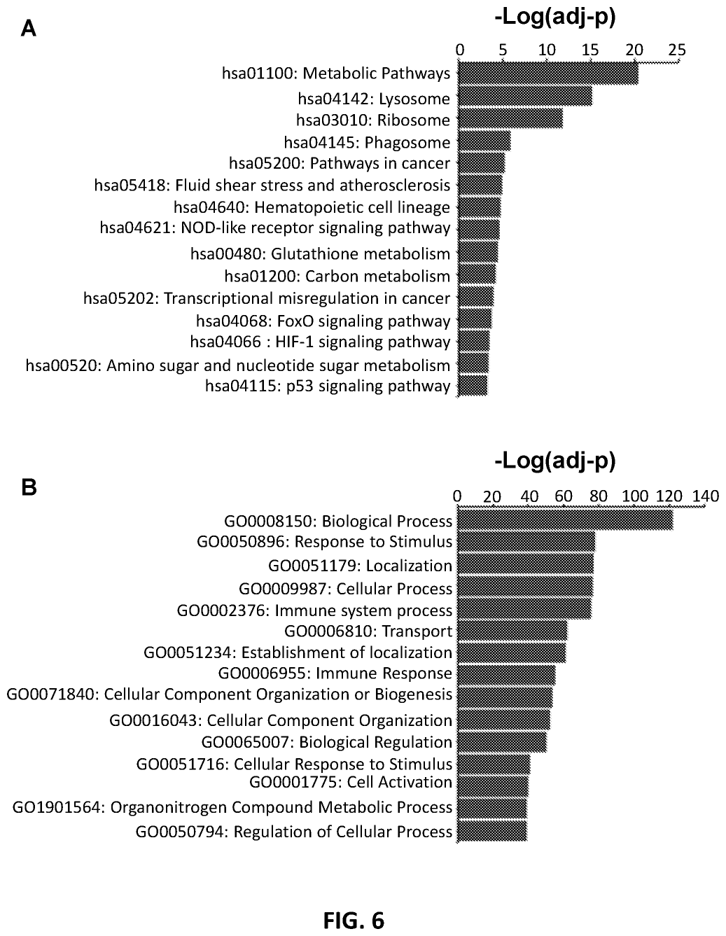

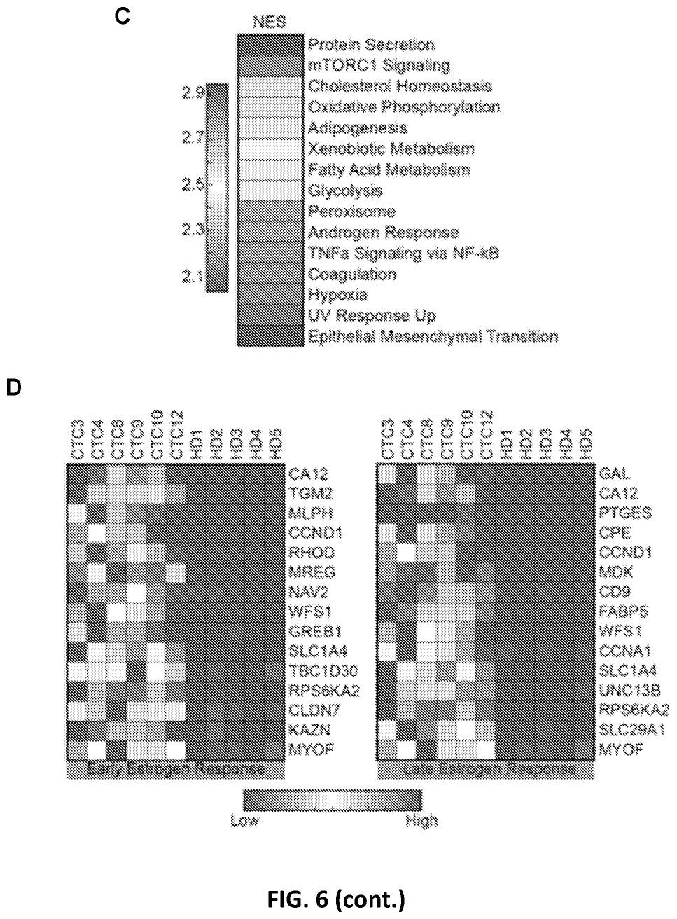

The RNA data were also analyzed using the Kyoto Encyclopedia of Genes and Genomes (KEGG) computational tool, which found the enrichment of 52 pathways (adjusted p-value <0.05; see FIG. 6A). The most significantly enriched (adjusted p-value=4.35×10⁻²¹) pathway for the dataset was KEGG pathway hsa01100, which encompasses all metabolic processes in the cell. KEGG analysis also detected other pathways more explicitly associated with cancer, including pathways in cancer (has05200) and transcriptional misregulation in cancer (hsa05202) (see FIG. 6A). Finally, KEGG analysis highlighted several signaling pathways associated with cancer progression and metastasis, including FoxO (hsa04068), p53 (hsa04115), and RAP1 (hsa04015) (see FIG. 6A).

The Gene Ontology (GO) project was used to identify affected biological processes based on the gene expression data. Notably, biological processes such as positive regulation of locomotion (GO:0040017, FDR=6.61×10⁻⁹) and cell migration (GO:0030335, FDR=5.94×10⁻⁹) suggest that propagated CTCs exhibit migratory properties consistent with the properties of metastatic cells (see FIG. 6B). Among the highly enriched processes were those associated with leukocyte activation (GO:0002274, FDR=7.35×10⁻¹⁸), and in particular neutrophils (GO:0042119, FDR=1.10×10⁻¹³), which supports the notion that leukocytes are present in the cultures.

Gene Set Enrichment Analysis (GSEA) was used to analyze the expression of gene sets to detecting multi-gene biological processes. This analysis identified 31 gene sets that were enriched in CTCs compared to HDs, including those associated with leukocyte activation (GO:0002274, FDR=7.35×10⁻¹⁸) and in particular neutrophils (GO:0042119, FDR=1.10×10-13). (see FIG. 6c). Gene sets associated with epithelial mesenchymal transition (normalized enrichment score (NES)=2.03), early (NES=1.60) and late (NES=1.52) estrogen response, which are biological properties of breast cancer and metastasis, were enriched in CTCs compared to HDs (see FIG. 6D).

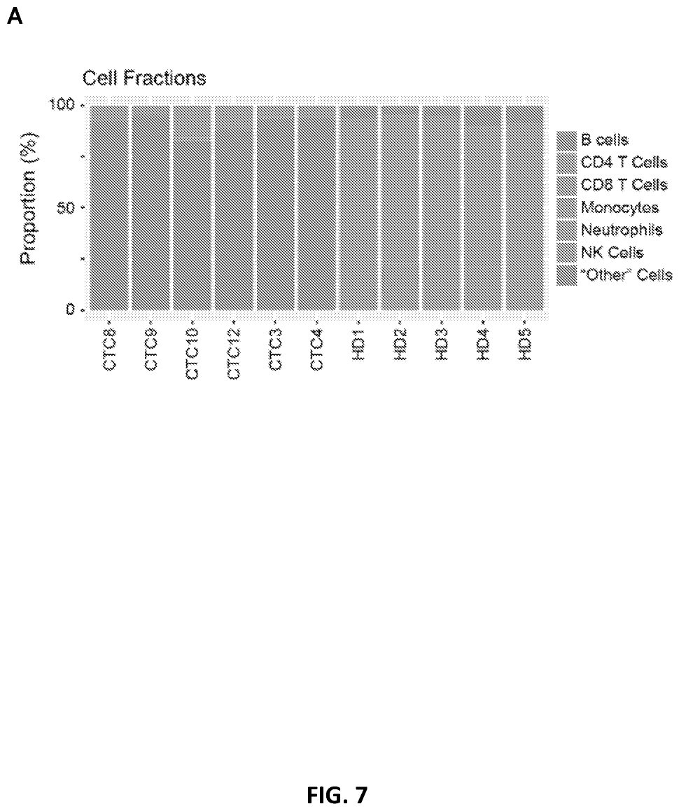

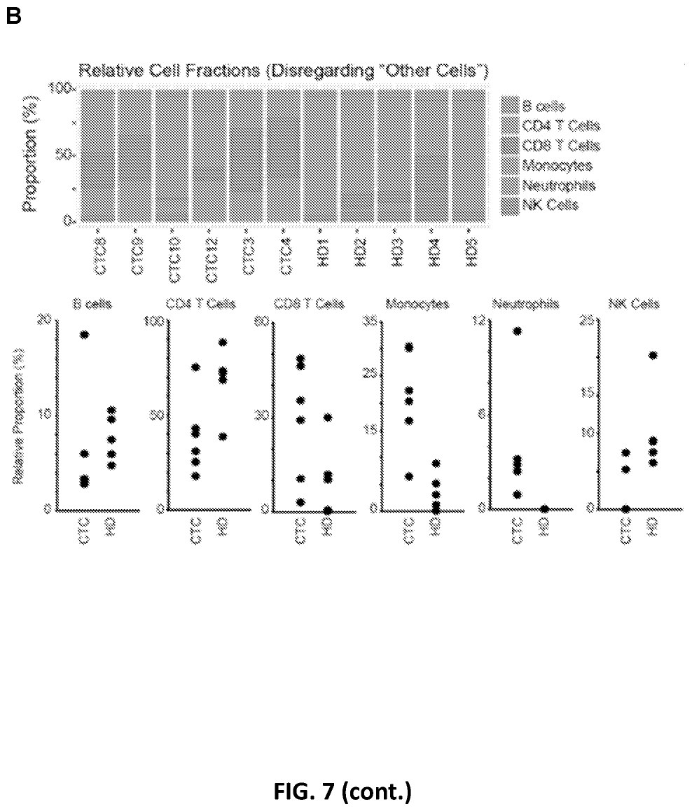

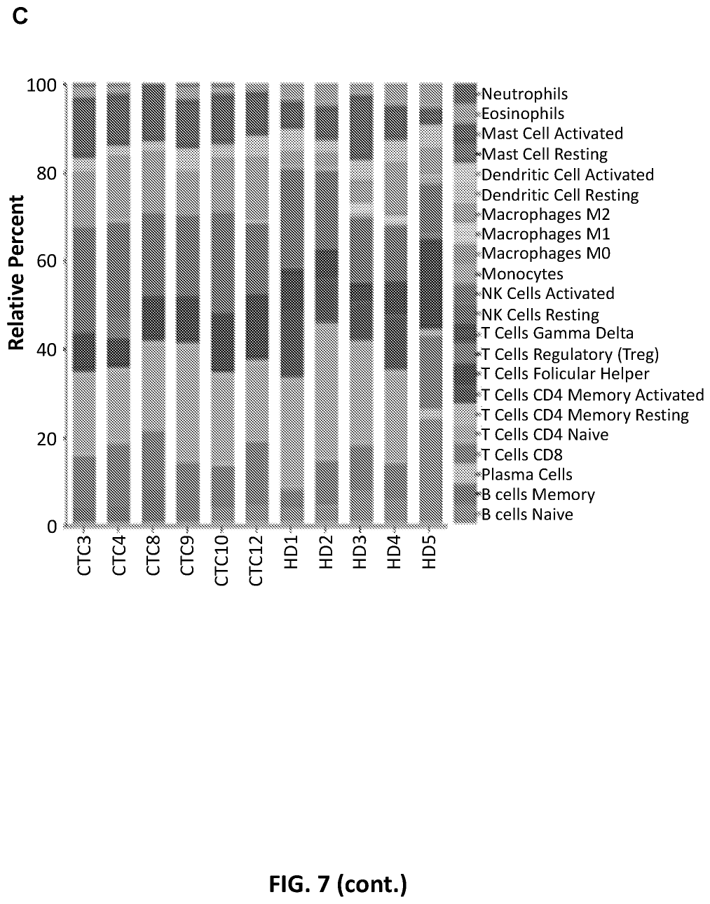

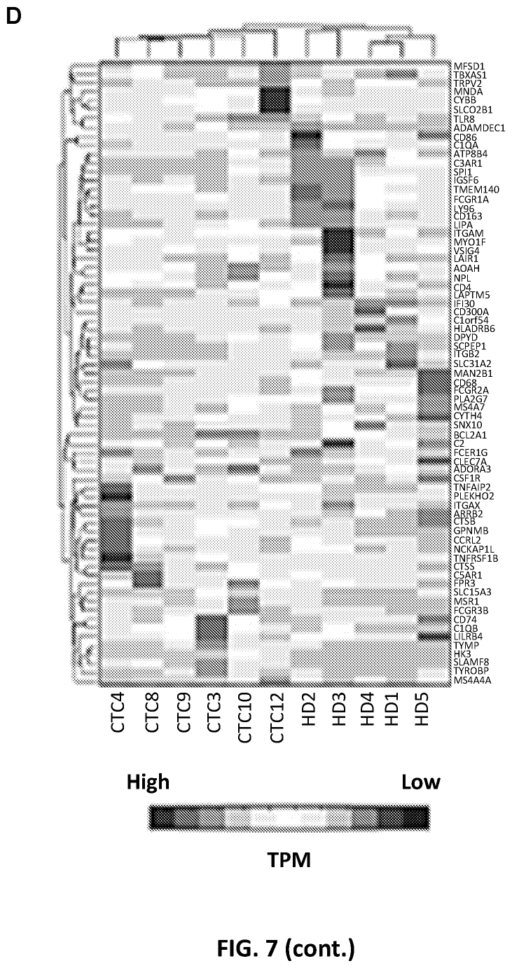

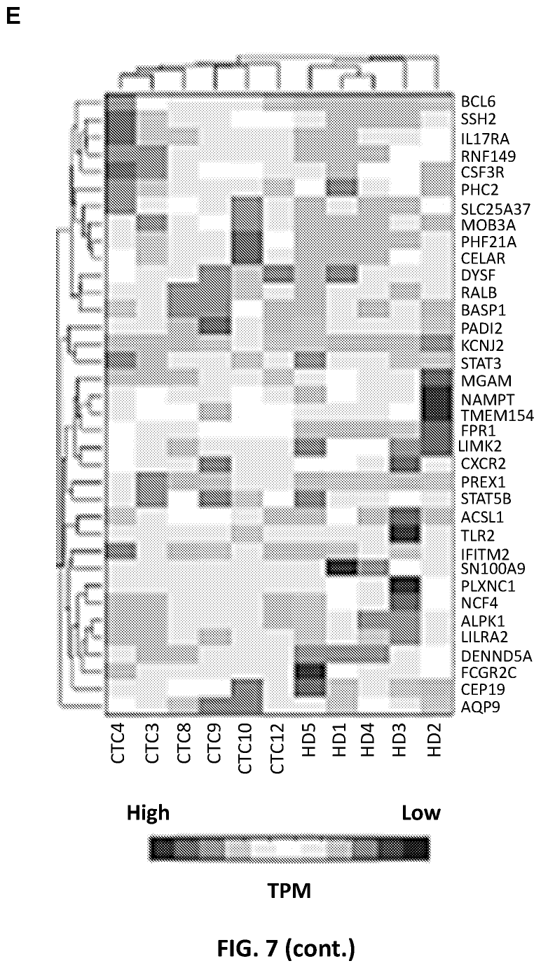

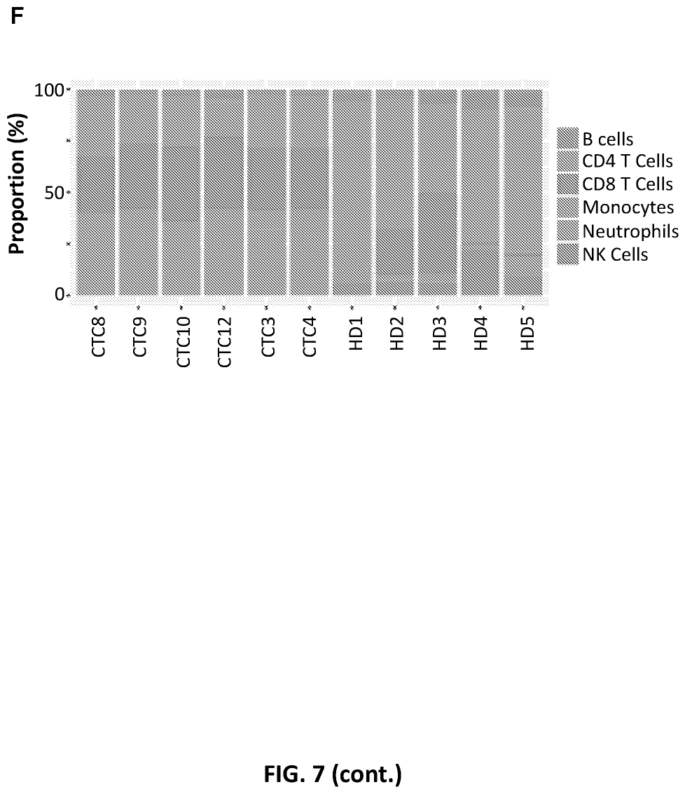

The results show that CTC associated with CD45⁺ cells result in longer term culture survival (and greater numbers of CTCs for experimental studies/clinical evaluation) (see FIGS. 4C-4E). Two computational tools—Estimating the Proportion of Immune and Cancer cells (EPIC) and CIBERSORT—was used to identify immune cell types based on gene expression data. EPIC determined that the majority of cells in the CTC cultures classified as “other”, i.e., of non-immunological origin (see FIG. 7A), which is consistent with the expansion of CTCs (of epithelial origin) and depletion of blood cells (including immune cells) during the culture conditions (see FIG. 4B). When only the immune cell populations were analyzed in EPIC, NK cells (2.1% in CTC vs 10.3% in HDs) and CD4 T cells (38.5% vs 67.9%) were depleted, while the proportion of neutrophils (3.5% vs 0.00002%), monocytes (20.9% vs 3.6%), and CD8 T cells (28.7% vs 10.4%) increased in CTC cultures vs HD samples (see FIG. 7B). Neutrophils showed the highest enrichment. Using CIBERSORT, CD8 T cells, neutrophils, and M2 macrophages were similarly enriched in CTC samples (see FIG. 7C). Finally, ImSig, an analytical tool used to identify immune cell sub-populations based on gene signatures, also validated these findings by showing that 17/35 genes were enriched in CTCs compared to HDs, including genes associated with macrophages and neutrophils (see FIG. 7E).

These results show that the methods according to embodiments of the present invention expand and enrich CTCs and, among leukocytes, favor the survival of neutrophils and macrophages.

### Example 2

A study was conducted to isolate and culture CTCs from patients with metastatic pancreatic cancer, in accordance with embodiments of the present invention. In the study, 10-20 ml of total blood from the patients was collected in two Li-heparinized tubes and immediately processed in the lab. Blood from each tube was measured and diluted 1:1 with HBSS (containing phosphate and magnesium, pH ˜7.4) solution in 50 ml sterile falcon tubes.

Blood was mixed well with HBSS before gently layering on the top of 15 ml of FICOLL-PAQUE™. Care was taken to not mix the FICOLL and the HBSS diluted blood. Both tubes were then gently placed in a centrifuge equipped with swinging bucket rotor and spun at 400 g for 40 min at 40° C. with minimal deceleration and acceleration.

Plasma and mononuclear layers from the top of the tubes were extracted and placed in a new 50 ml Falcon tube and diluted with HBSS to 45 ml. Tubes were centrifuged at 400 g for 10 min at 18° C. After spinning, the supernatant was discarded, and the cell pellet was resuspended with 5 ml HBSS and gently spun down at 400 g for 10 min.

The resultant cell pellet was washed twice with 10 ml PBS, without calcium and magnesium. After washing, the cell pellet was then resuspended gently in 3DCC media and placed in a low-attachment 6-cm plate and incubated at 37° C. in an incubator equipped with about 2% oxygen and 5% carbon dioxide. The 3DCC medium comprised a mixture of DMEM and F12, B-27, an antibiotic and anti-fungal mix, 20 ng/ml EGF, 20 ng/ml bFGF, 10 μg/ml Heparin, 10 μM Y-compound and 25 μg/ml adenine.

CTCs were successfully established in the 3DCC.

### Example 3

A study was conducted to isolate and culture CTCs from patients with metastatic lung cancer, both small cell lung cancer and non-small cell lung cancer, in accordance with embodiments of the present invention. In the study, 10-20 ml of total blood from the patients was collected in two Li-heparinized tubes and immediately processed in the lab. Blood from each tube was measured and diluted 1:1 with HBSS (containing phosphate and magnesium, pH ˜7.4) solution in 50 ml sterile falcon tubes. CTCs were then isolated and cultured in accordance with the methods described in Example 2.

CTCs were successfully established in the 3DCC.

### Example 4

A study was conducted to isolate and culture CTCs from patients with metastatic colorectal cancer, in accordance with embodiments of the present invention. In the study, 10-20 ml of total blood from the patients was collected in two Li-heparinized tubes and immediately processed in the lab. Blood from each tube was measured and diluted 1:1 with HBSS (containing phosphate and magnesium, pH ˜7.4) solution in 50 ml sterile falcon tubes. CTCs were then isolated and cultured in accordance with the methods described in Example 2.

CTCs were successfully established in the 3DCC.

### Example 5

A study was conducted to generate tumors by injecting into mice CTCs obtained from samples described in Examples 3 and 4. These samples are summarized in Table 1 below, which shows that they are from a patient with metastatic lung cancer (identified as sample 19), from a first patient with metastatic colorectal cancer (sample 21), or from a second patient with metastatic colorectal cancer (sample 22).

For each sample, the CTCs were grown in 3DD for more than 30 days. They were mixed with 50 μl PBS and 50 μl growth factor reduced matrigel, and were injected subcutaneously into the flank the mouse. Two mice were injected with CTCs from sample 19, one mouse was injected with CTCs from sample 21, and two mice were injected with CTCs from sample 22 (see Table 1).

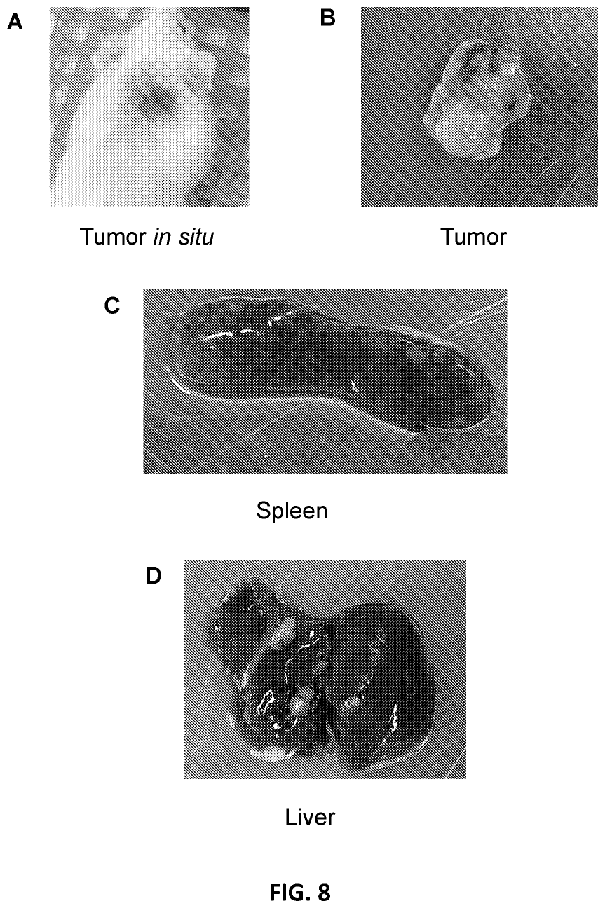

Palpable tumors were observed within 27 days in the mice injected with CTCs from sample 19 or sample 21. FIG. 8A shows an example of the tumor in situ in the scruff of the neck of a mouse. FIG. 8B shows an example of a tumor that was removed from a mouse.

Tumors were measured every week and harvested when they reached a size of about 17 mm×15 mm. The tumors showed extensive necrosis and angiogenesis, as seen with the red patch on the neck of the mouse (FIG. 8A). In addition, mice showed macro-metastasis in various organs including spleen (FIG. 8C), liver (FIG. 8D), bones, and lymph node.

These results demonstrate the metastatic tumorigenic phenotype of the CTCs that were obtained from the patients.

TABLE 1
Summary of the samples used to generate tumors in
mice injected with CTCs and the outcome of the rejection.

| | | Number of | Days to | |
|---|---|---|---|---|
| CTC | Patient | Animals | Palpable | Tumor |
| Sample | Diagnosis | Injected | Tumor | Measurement |
| 19 | | 2 | 27 days | 17.0 mm × 15.0 mm |
| | Metastatic lung | | | 11.2 mm × 9.0 mm |
| | cancer | | | |
| 21 | Metastatic | 1 | 27 days | 12.1 mm × 11.1 mm |
| | colorectal | | | |
| | cancer | | | |
| 22 | Metastatic | 2 | 45 days in | Non-measurable |
| | colorectal | | one mouse | Non-measurable |
| | cancer | | | |

### Example 6

A study was conducted to establish CTC-derived xenograft (CDX) mice models using cultured CTCs injected into immunocompromised mice at subcutaneous site into the flank. The CTCs were mixed with PBS:matrigel (1:1).

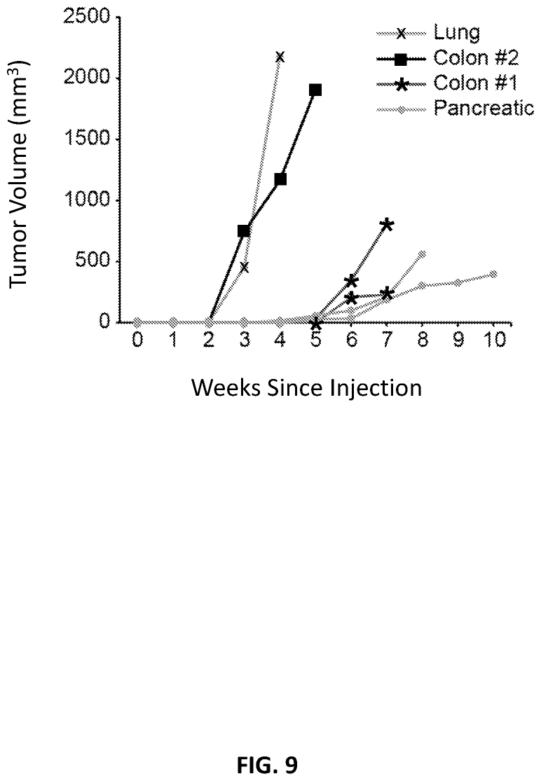

CDX models were generated for metastatic lung, breast, colon, and pancreatic tumors. Mice were observed for palpable tumor growth and the results are shown in FIG. 9 for four individual metastatic CDX models of lung, colon (two), and pancreatic cancer. Table 2 shows the macro-metastasis pattern in the CDX models.

TABLE 2
Summary of the macro-metastasis pattern in the CDX models.

| | | Lymph | | | |
|---|---|---|---|---|---|
| Sample | Lung | Node | Spleen | Liver | Ovary |
| Colon #1 | No | Yes | Yes | Yes | No |
| Colon #2 | Yes | Yes | Yes | Yes | No |
| Lung #1 | No | Yes | Yes | No | No |
| Pancreas #1 | No | Yes | Yes | No | No |
| Breast #1 | No | Yes | No | No | Yes |

Yes = macro-metastasis was present
No = no macro-metastasis was present

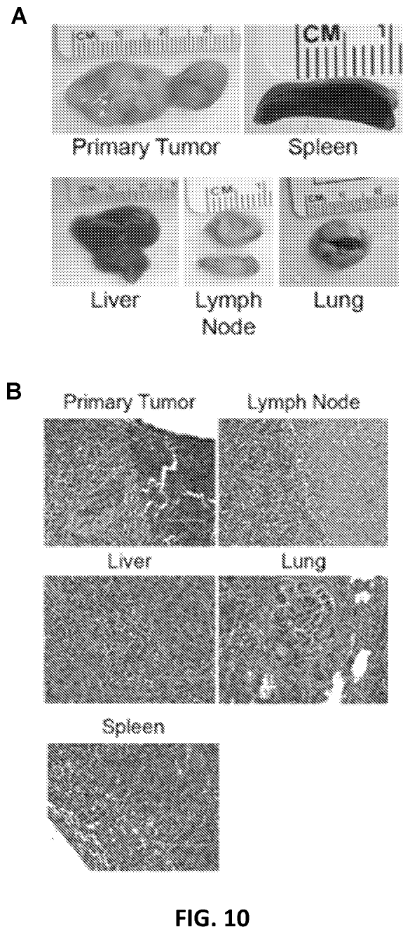

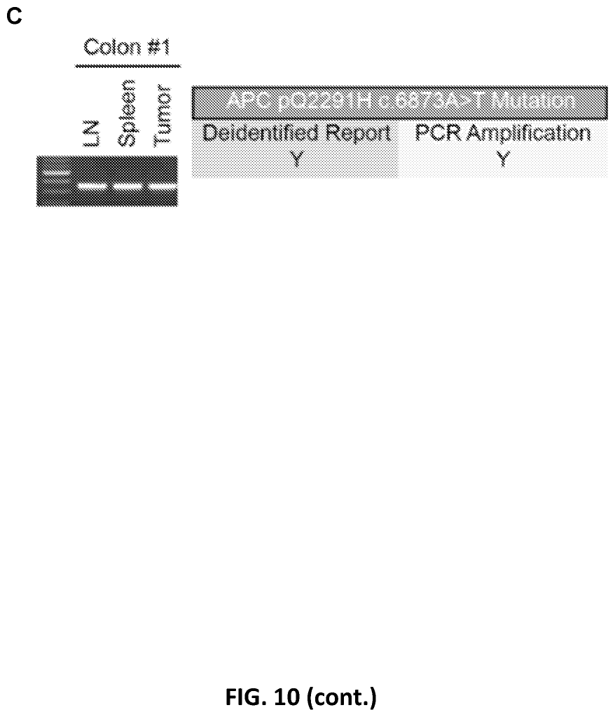

When the primary tumor reached the maximum size allowed mice were euthanized and primary tumor was harvested along with several macro-metastatic tumor tissues. In one of the colon cancer CDX models, macro-metastases were observed in the spleen, liver, lung, and lymph nodes (see FIG. 10A). Tissues were fixed in formalin and subsequently embedded in paraffin. Representative images of Hematoxylin and Eosin (H&E) images revealed un-differentiated tumor morphology as shown in FIG. 10B. Mutation in adenomatous polyposis coli (APC) gene in 30% of primary tumor cells were recorded, which suggested a tumor-specific mutation. The presence of this mutation was confirmed in CDX primary tumor and corresponding metastases with real-time polymerase chain reaction (PCR) using specific primer sets flanking the mutation site followed by Sanger sequencing of the PCR product (see FIG. 10C).

The foregoing description is given for clearness of understanding only, and no unnecessary limitations should be understood therefrom, as modifications within the scope of the invention may be apparent to those having ordinary skill in the art.

Throughout this specification and the claims that follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A method of generating a population of tumor cells isolated from a subject, the method comprising
(a) collecting a fluid sample containing the tumor cells from the subject;
(b) culturing the tumor cells in a three-dimensional cell culture, wherein the three-dimensional cell culture comprises at least one inhibitor of Rho-kinase (ROCK inhibitor), to generate the population of tumor cells.

2. The method of claim 1, wherein the fluid sample comprises whole blood, pleural effusion, pericardial effusion, ascites, cerebrospinal fluid, urine, fluid from ductal lavage, fluid from bronchoalveolar lavage, fluid from endoscopic retrograde cholangiopancreatography duct cytologic brushing, fluid from esophageal brushing, fluid from cervical brushing, fluid from uterine brushing, or fluid from cystic lesions.

3. The method of claim 1 or 2, wherein the subject has abnormal epithelial tissue.

4. The method of claim 3, wherein the abnormal epithelial tissue is an epithelial malignancy, in situ tumor, premalignant lesion, or benign lesion.

5. The method of any one of claims 1-4, further comprising isolating the tumor cells prior to culturing the tumor cells in the three-dimensional cell culture.

6. The method of claim 5, wherein isolating the tumor cells is by density gradient separation.

7. The method of claim 5, wherein isolating the tumor cells is by centrifugation.

8. A method of generating a population of circulating tumor cells (CTCs) isolated from a subject, the method comprising
(a) collecting a whole blood sample from the subject;
(b) subjecting the collected whole blood sample to density gradient separation to separate the CTCs from at least erythrocytes in the whole blood sample; and
(c) culturing the separated CTCs in a three-dimensional cell culture, wherein the three-dimensional cell culture comprises at least one inhibitor of Rho-kinase (ROCK inhibitor), to generate the population of CTCs

9. The method of claim 8, wherein the CTCs are derived from one or more malignant tumors in the subject.

10. The method of claim 8 or 9, wherein the CTCs do not originate from a blood-born cancer.

11. The method of any one of claims 8-10, wherein the whole blood sample is collected in a heparinized collection tube.

12. The method of any one of claims 8-11, wherein the whole blood sample is diluted with a saline solution prior to being subjected to density gradient separation.

13. The method of any one of claims 8-12, wherein the density gradient separation comprises a medium selected from the group consisting of a polysaccharide, colloidal silica particles, a polyhydric alcohol, inorganic salt, and iodinated gradient media.

14. The method of any one of claims 8-13, wherein the density gradient separation separates the CTCs from free granulocytes in the whole blood sample.

15. The method of any one of claims 1-14, wherein the three-dimensional cell culture comprises a cell scaffold selected from the group consisting of a hydrogel, collagen sponge, gelatin, nanofiber scaffold, polycaprolactone scaffolds, and polystyrene.

16. The method of any one of claims 1-15, wherein the three-dimensional cell culture comprises a scaffold-free vessel.

17. The method of claim 1-16, wherein the three-dimensional cell culture comprises a culture vessel that inhibits attachment of the cells to the vessel surface.

18. The method of any one of claims 1-17, wherein the at least one ROCK inhibitor is a small molecule inhibitor of Rho kinase 1 (ROCK 1), a small molecule inhibitor Rho kinase 2 (ROCK 2), a RNAi molecule directed to ROCK 1, a RNAi molecule directed to ROCK 2, or a combination thereof.

19. The method of any one of claims 1-18, wherein the at least one ROCK inhibitor is selected from the group consisting of Y-27632, HA1100 hydrochloride, HA1077, GSK429286, or a combination thereof.

20. The method of any one of claims 1-19, wherein the three-dimensional cell culture further comprises adenine.

21. The method of any one of claims 1-20, wherein the three-dimensional cell culture is serum free.

22. The method of any one of claims 1-21, wherein the three-dimensional cell culture comprises oxygen in an amount of less than about 10%.

23. The method of any one of claims 1-22, wherein the three-dimensional cell culture comprises oxygen in an amount of less than about 5%.

24. The method of any one of claims 1-23, wherein the three-dimensional cell culture comprises carbon dioxide in an amount of less than about 10%.

25. The method of any one of claims 1-24, wherein the three-dimensional cell culture comprises feeder cells.

26. The method of any one of claims 1-25, wherein the method does not include a cell-surface marker separation technique.

27. The method of any one of claims 1-26, wherein the method does not include the use of microfluidics.

28. A method of identifying a candidate treatment for a subject having a condition marked by the presence of tumor cells, the method comprising
(a) obtaining a population of the tumor cells, wherein the population of the tumor cells is generated by:
(i) collecting a fluid sample containing the tumor cells from the subject;
(ii) culturing the tumor cells in a three-dimensional cell culture, wherein the three-dimensional cell culture comprises at least one inhibitor of Rho-kinase (ROCK inhibitor), to generate the population of tumor cells;
(b) determining a response profile of at least a portion of the population of tumor cells; and
(c) identifying a candidate treatment for the subject based on the determined response profile.

29. The method of claim 26, further comprising isolating the tumor cells prior to culturing the tumor cells in the three-dimensional cell culture.

30. A method of identifying a candidate treatment for a subject having a condition marked by the presence of tumor cells, wherein the tumor cells are circulating tumor cells (CTCs), the method comprising
(a) obtaining a population of the CTCs, wherein the population of the CTCs is generated by:
(i) collecting a whole blood sample from the subject;
(ii) subjecting the collected whole blood sample to density gradient separation to separate the CTCs from at least erythrocytes in the whole blood sample; and
(iii) culturing the separated CTCs in a three-dimensional cell culture, wherein the three-dimensional cell culture comprises at least one inhibitor of Rho-kinase (ROCK inhibitor), to generate the population of CTCs;
(b) determining a response profile of at least a portion of the population of CTCs; and
(c) identifying a candidate treatment for the subject based on the determined response profile.

31. The method of any one of claims 28-30, wherein the condition marked by the presence of tumor cells comprises having one or more neoplasias, hyperplasias, malignant tumors, in situ tumors, benign tumors, or a combination thereof.

32. The method of any one of claims 28-31, wherein the response profile is at least partially determined by identifying the sequence of at least one portion of DNA extracted from the tumor cells.

33. The method of any one of claims 28-31, wherein the response profile is at least partially determined by identifying at least one mRNA that is produced in the tumor cells.

34. The method of any one of claims 28-31, wherein the response profile is at least partially determined by identifying at least one mRNA that is not produced in the tumor cells.

35. The method of any one of claims 28-31, wherein the response profile is at least partially determined by identifying one or more proteins that the tumor cells express.

36. The method of any one of claims 28-31, wherein the response profile is at least partially determined by identifying one or more proteins that the tumor cells do not express.

37. The method of any one of claims 28-31, wherein the response profile is at least partially determined by subjecting the tumor cells to a pharmacologic therapy and determining the therapeutic index of the chemotherapeutic agent on the tumor cells.

38. The method of claim 37, wherein the pharmacologic therapy comprises one or more chemotherapeutic agents, one or more targeted agents, one or more endocrine agents, one or more immunotherapies, or a combination thereof.

39. A method of monitoring in a subject the persistence, regression, or progression of a disease or condition marked by the presence of tumor cells, the method comprising
(a) obtaining a population of the tumor cells, wherein the population of the tumor cells is generated by:
(i) collecting a fluid sample containing the tumor cells from the subject;
(ii) culturing the tumor cells in a three-dimensional cell culture, wherein the three-dimensional cell culture comprises at least one inhibitor of Rho-kinase (ROCK inhibitor), to generate the population of tumor cells;
(b) assaying at least a portion of the population of tumor cells; and
(c) evaluating the results of the assay to determine whether the disease has persisted, regressed, or progressed.

40. The method of claim 39, further comprising isolating the tumor cells prior to culturing the tumor cells in the three-dimensional cell culture.

41. A method of monitoring in a subject the persistence, regression, or progression of a disease or condition marked by the presence of tumor cells, wherein the tumor cells are circulating tumor cells (CTCs), the method comprising
(a) obtaining a population of the CTCs, wherein the population of the CTCs is generated by:
(i) collecting a whole blood sample from the subject;
(ii) subjecting the collected whole blood sample to density gradient separation to separate the CTCs from at least erythrocytes in the whole blood sample; and
(iii) culturing the separated CTCs in a three-dimensional cell culture, wherein the three-dimensional cell culture comprises at least one inhibitor of Rho-kinase (ROCK inhibitor), to generate the population of CTCs;
(b) assaying at least a portion of the population of CTCs; and
(c) evaluating the results of the assay to determine whether the disease has persisted, regressed, or progressed.

42. The method of any one of claims 39-41, wherein the condition marked by the presence of tumor cells comprises having one or more neoplasias, hyperplasias, in situ tumors, malignant tumors, benign tumors, or a combination thereof.

43. The method of any one of claims 39-42, wherein the at least a portion of the population of tumor cells is assayed to identify the presence, or the extent to which there is a presence, of a marker of persistence, regression, or progression.

44. The method of any one of claim 39-43, wherein the assay comprises identifying the sequence of at least one portion of DNA extracted from the tumor cells.

45. The method of any one of claim 39-43, wherein the assay comprises identifying at least one mRNA that is produced in the tumor cells.

46. The method of any one of claim 39-43, wherein the assay comprises identifying at least one mRNA that is not produced in the tumor cells.

47. The method of any one of claim 39-43, wherein the assay comprises identifying one or more proteins that the tumor cells express.

48. The method of any one of claim 39-43, wherein the assay comprises identifying one or more proteins that the tumor cells do not express.

49. A method of generating a cell line of tumor cells isolated from a subject, the method comprising
(a) collecting a fluid sample containing the tumor cells from the subject;
(b) culturing the tumor cells in a three-dimensional cell culture, wherein the three-dimensional cell culture comprises at least one inhibitor of Rho-kinase (ROCK inhibitor), to generate the population of tumor cells.

50. The method of claim 49, further comprising isolating the tumor cells prior to culturing the tumor cells in the three-dimensional cell culture.

51. A method of generating a cell line of circulating tumor cells (CTCs) isolated from a subject, the method comprising
(a) collecting a whole blood sample from the subject;
(b) subjecting the collected whole blood sample to density gradient separation to separate the CTCs from at least erythrocytes in the whole blood sample; and
(c) culturing the separated CTCs in a three-dimensional cell culture, wherein the three-dimensional cell culture comprises at least one inhibitor of Rho-kinase (ROCK inhibitor).

52. A cell culture composition comprising (a) a population of tumor cells, and (b) a three-dimensional cell culture (3DCC) comprises at least one inhibitor of Rho-kinase (ROCK inhibitor).

53. The cell culture composition of claim 47, wherein the population of tumor cells is generated by:
(a) collecting a fluid sample containing the tumor cells from the subject;
(b) culturing the tumor cells in the 3DCC comprising the at least one ROCK inhibitor, to generate the population of tumor cells.

54. The cell culture composition of claim 53, further comprising isolating the tumor cells prior to culturing the tumor cells in the three-dimensional cell culture.

55. A cell culture composition comprising (a) a population of circulating tumor cells (CTCs), and (b) a three-dimensional cell culture (3DCC) comprises at least one inhibitor of Rho-kinase (ROCK inhibitor).

56. The cell culture composition of claim 49, wherein the population of CTCs is generated by:
(a) collecting a whole blood sample from the subject;
(b) subjecting the collected whole blood sample to density gradient separation to separate the CTCs from at least erythrocytes in the whole blood sample; and
(c) culturing the separated CTCs in a three-dimensional cell culture, wherein the three-dimensional cell culture comprises at least one inhibitor of Rho-kinase (ROCK inhibitor), to generate the population of CTCs.

57. The method or cell culture composition of any one of claims 1-56, wherein the three-dimensional cell culture further comprises transferrin, insulin, and putrescine.

58. A method of generating a population of tumor cells isolated from a subject, the method comprising
(a) collecting a fluid sample containing the tumor cells from the subject;
(b) culturing the tumor cells in a three-dimensional cell culture, wherein the three-dimensional cell culture comprises:
(i) at least one inhibitor of Rho-kinase (ROCK inhibitor),
(ii) transferrin,
(iii) insulin, and
(iv) putrescine,
to generate the population of tumor cells.

59. The method of claim 58, wherein the tumor cells are circulating tumor cells (CTCs).

60. The method of claim 58 or 59, wherein the fluid sample is a whole blood sample.

61. The method of claim 60, further comprising subjecting the collected whole blood sample to density gradient separation to separate the CTCs from at least erythrocytes in the whole blood sample, prior to culturing the tumor cells in the three-dimensional cell culture.

62. A cell culture composition comprising (a) a population of tumor cells, and (b) a three-dimensional cell culture comprising at least one inhibitor of Rho-kinase, transferrin, insulin, and putrescine.
